(19) United States
(12) Patent Application Publication
(10) Pub. No.: US 2025/0201335 A1
(43) Pub. Date: Jun. 19, 2025

(54) METHODS AND SYSTEMS FOR ANALYZING CHROMATINS

(71) Applicants: LUDWIG INSTITUTE FOR CANCER RESEARCH LTD, Zurich (CN); THE REGENTS OF THE UNIVERSITY OF CALIFORNIA, Oakland, CA (US)

(72) Inventors: Bing Ren, La Jolla, CA (US); Bojing Blair Jia, La Jolla, CA (US)

(21) Appl. No.: 18/847,819

(22) PCT Filed: Mar. 17, 2023
(86) PCT No.: PCT/US23/64607

§ 371 Date: Sep. 17, 2024

Related U.S. Application Data
(60) Provisional application No. 63/321,349, filed on Mar. 18, 2022.

(51) Int. Cl. G16B 15/10; C12Q 1/6841; G16B 20/10; G16B 40/10

(52) U.S. Cl. CPC G16B 15/10; C12Q 1/6841; G16B 20/10; G16B 40/10

(57) ABSTRACT

This disclosure provides a novel method and system using a “spatial genome aligner” that parses true chromatin signals from noise by aligning signals to a DNA polymer model. This spatial genome aligner can efficiently reconstruct chromosome architectures from DNA-fluorescence in situ hybridization (DNA-FISH) data across multiple scales and determine chromosome ploidies de novo in interphase cells.

### CROSS-REFERENCE TO RELATED APPLICATIONS

This application is a national phase of International Patent Application No. PCT/US23/64607 filed Mar. 17, 2023, which claims the benefit of priority to U.S. Provisional Patent Application Ser. No. 63/321,349 filed Mar. 18, 2022, the disclosures of which are hereby incorporated by reference in their entiretyies.

### STATEMENT REGARDING FEDERALLY SPONSORED RESEARCH

This invention was made with government support under 5UM1HG011585 awarded by the National Institutes of Health and National Human Genome Research Institute, and under GM008666 by the Ruth Kirschstein Institutional National Research Service Award. The government has certain rights in the invention.

### FIELD OF THE INVENTION

This invention relates to methods and systems for analyzing chromatins.

### BACKGROUND OF THE INVENTION

Eukaryotic chromosomes undergo dramatic compaction and decompaction in the life cycle of a cell, and the dynamic chromosomal structure plays an integral role in a range of nuclear processes such as DNA replication, recombination, repair, and gene transcription. In interphase nuclei, different chromosomes generally occupy separate territories with limited intermingling. Within each chromosomal territory, the chromatin fibers are organized into compartments and domains, driven in part by the ATP-dependent motor protein complex and loop extruder cohesin. The complex chromatin structures enable juxtaposition of remote DNA in space and subsequent transcriptional activation of genes by distal enhancers. Disruption of chromatin structures underlies a score of pathologies such as limb malformations, oncogenesis, and heart disease. Delineating how chromatin fibers are folded in the nucleus is therefore of fundamental importance for study of gene regulation and other nuclear processes in health and disease.

Multiplexed DNA Fluorescence In situ Hybridization (M-DNA-FISH) has emerged recently as a powerful imaging technique for the study of chromatin structure in eukaryotic cells. These technologies utilize serial hybridization of fluorescent probes to tens to thousands of genomic loci in the nucleus to detect the folding patterns of chromosomes. By design, fluorescent probes label discrete genomic loci; the chromatin fiber connecting them is not visualized and must be inferred. The inference of physical connection between two discrete signals is the most salient problem facing chromatin imaging to date. Early efforts to multiplex DNA-FISH often found one, three, or four fluorescent signals emanating from one genomic region in a diploid cell line expected to produce two signals. Biological copy number variation, chromosomal intermingling as well as poor probe hybridization have been acknowledged to explain missing signals, and sister chromatids and aneuploidy as well as imaging noise have been acknowledged to explain extra signals. If both noise and biological variation can explain any observed scenario, chromatin fibers cannot be naïvely traced by connecting the first immediate spot. In fact, this uncertainty around imaging has led some to forgo tracing altogether and instead tabulate proximal pairs of imaged loci for bulk analysis. When noise appears indistinguishable from true imaged genomic loci, and biological variation at the single cell level confounds expectation, reconstruction of chromatin fibers remains an intractable computational problem.

In the current benchmark for chromatin tracing, the tracing problem is simplified with assumptions about copy number and emphasize the optical quality of detected signals. Explicitly, an expectation-maximization algorithm (E-M) is first tasked to find k chromatin fibers corresponding to a k-ploid cell. Repeated k-times per cell, candidate fluorescence spots corresponding to a genomic region are scored based on signal intensity, proximity to a moving average of downstream selected spots as well as upstream selected spots, and proximity to a chromosome center (i.e., the aggregate of many fluorescent loci) determined by k-means clustering. Implicit in this approach are two strong assumptions: that the brightness is a measure of detection confidence and that the copy number of a DNA segment is fixed and known beforehand. However, background fluorescence, non-specific probe binding, and even hot pixels can frequently emit similarly intense focal signals indistinguishable from the true signal. Additionally, looking for a fixed number of chromosomes may fail to capture true biological copy number variations and aneuploidy.

Accordingly, there exists a strong need for improved methods and systems for analyzing chromatins.

### SUMMARY OF THE INVENTION

This disclosure addresses the need mentioned above in a number of aspects. In one aspect, this disclosure provides a method for analyzing chromatins, comprising:
(a) obtaining a fluorescence imaging dataset comprising a three-dimensional image stack generated using a plurality of fluorescent probes hybridizing to discrete genomic loci on one or more chromatins, wherein the image stack comprises a plurality of fluorescence signals, each corresponding to a set of fluorescent probes of the plurality of fluorescent probes;
(b) associating a plurality of nodes respectively with the plurality of fluorescence signals, wherein one node is assigned to one fluorescence signal;
(c) assigning a locus order to each of the nodes according to the genomic coordinate on a reference genome of each of the nodes such that one or more candidate nodes are associated with each locus order, and assigning coordinates corresponding to spatial coordinates of a genomic locus detected in fluorescence imaging to define a spatial position of each of the nodes;
(d) connecting a first candidate node of a first locus order with a second candidate node of a second locus order to form an edge, wherein the second locus order is greater than the first locus order by one or more locus orders;
(e) determining an edge weight based on a DNA polymer model to define a probability of the edge being an actual physical connection between two genomic loci represented by the first candidate node and the second candidate node on the reference genome;
(f) repeating steps (d) to (e) for remaining candidate nodes of the first locus order and remaining candidate nodes of the second locus order;
(g) traversing candidate nodes of remaining locus orders by repeating steps (d) to (f) to form a plurality of paths, each representing a spatial configuration of a potential chromatin fiber;
(h) determining a sum of edge weights of all the edges traversed in each of the paths, wherein the sum of edge weights defines a physical likelihood of the potential chromatin fiber; and
(i) identifying one or more potential chromatin fibers having the sum of edge weights greater than a physical likelihood threshold.

In some embodiments, determining the edge weight comprises comparing observed pairwise spatial distance between the first candidate node and the second candidate node with estimated pairwise spatial distance between the two genomic loci represented by the first candidate node and the second candidate node on a reference chromatin fiber.

In some embodiments, the estimated pairwise spatial distance between the two genomic loci on the reference chromatin fiber is calculated using a freely joined Gaussian chain model.

In some embodiments, the edge weight is determined by:

$${w}_{t ; i}^{t + c ; j} = \frac{1}{{( 2 {π ( {S}_{t ; i}^{t + c ; j} )}^{2} )}^{3 / 2}} {e}^{- ( \frac{{( {R}_{t ; i}^{t + c ; j} )}^{2}}{2 {( {S}_{t ; i}^{t + c ; j} )}^{2}} )}$$

where R_{t;i}^{t+c;j} is a distance in nanometers between the ith node with locus order t to the jth node with locus order t+c;
wherein S_{t;i}^{t+c;j} is expanded as:

$${( {S}_{t ; i}^{t + c ; j} )}^{2} = {σ}_{t ; i}^{2} + {σ}_{t + c ; j}^{2} + \frac{2}{3} {l}_{p} τ {L}_{t ; i}^{t + c ; j}$$

where positional uncertainties of both the start locus σ_{t;i}² and end locus σ_{t+c;j}² are appended to the second moment

$$〈 {R}^{2} 〉 = \frac{2}{3} {l}_{p} τ {L}_{t ; i}^{t + c ; j} ,$$

where lₚ is persistence length of DNA in nanometers, x is a scaling factor that converts genomic distance in base pairs to spatial distance in nanometers, τ and L_{t;i}^{t+c;j} is the genomic distance in base pairs that separate the start locus v_{t;i} and end locus v_{t+c;j}.

In some embodiments, determining the edge weight comprises transforming the probability of the edge with a negative logarithm function into positive edge weights.

In some embodiments, the physical likelihood of the potential chromatin fiber is defined by:

$$CDF = {\prod }_{h = 1}^{H} {w}_{{v}_{h}}^{{v}_{h + 1}} , p = 〈 {v}_{1} , {v}_{2} … {v}_{H} 〉$$

for every node v visited on path p from source to sink, wherein CDF represents conformational distribution function which defines the physical likelihood.

In some embodiments, the method comprises ranking physical likelihoods of the potential chromatin fibers and identifying a potential chromatin fiber having the maximum physical likelihood.

In some embodiments, the method comprises finding the shortest path from a starting node of the first locus order to an ending node of an end locus order for the genomic loci on the reference genome. In some embodiments, the method comprises generating an adjacency matrix for finding the shortest path. In some embodiments, finding the shortest path is performed by dynamic programming. In some embodiments, the dynamic programming comprises performing a Dijkstra operation to find a least-cost path.

In some embodiments, the method comprises at step (c) assigning the coordinates corresponding to the spatial coordinates of the genomic locus comprises assigning to each of the nodes positional uncertainty in each spatial axis discovered from three-dimensional gaussian fitting.

In some embodiments, the second locus order is not immediately adjacent to the first locus order such that one or more intervening locus orders are skipped for edge connection. In some embodiments, the method comprises applying a gap penalty for the one or more intervening locus orders skipped.

In some embodiments, the fluorescence imaging dataset is obtained from a fluorescence in situ hybridization (FISH) procedure selected from sequential fluorescent in situ hybridization (seqFISH+), single-molecule fluorescent in situ hybridization (smFISH), multiplexed error-robust fluorescence in situ hybridization (MERFISH), multiplexed DNA fluorescence in situ hybridization (M-DNA-FISH), and whole-genome DNA seqFISH+ imaging.

In some embodiments, the fluorescence imaging dataset is obtained from the fluorescence in situ hybridization (FISH) procedure on a eukaryotic cell. In some embodiments, the discrete genomic loci have an interval of about 1 kb to about 10 Mb. In some embodiments, the discrete genomic loci can have unidentical, nonuniform intervals (e.g., 1.1 Mb→2.5 Mb→0.6 Mb→3.2 Mb→ . . . ) that span a chromosome. In some embodiments, the sequence of unidentical, nonuniform intervals can be appropriated as a spatial barcode, analogous to alternating black and white stripes of different widths in traditional barcodes.

In some embodiments, the method comprises: prior to step (i), (1) accepting all the potential chromatin fibers, (2) performing an iterative search wherein nodes of each shortest path discovered are subtracted and rendered unavailable for other path traversals before searching for the next shortest path, until no likely paths below the physical likelihood threshold remain to be discovered, and (3) counting the number of all physically likely potential chromatin fibers.

In some embodiments, the method comprises performing clustering (e.g., k-means clustering) on one or more potential chromatin fibers to determine a spatial distribution of the one or more potential chromatin fibers in one or more locations of chromosome territory.

In some embodiments, the method comprises performing density-based clustering on the one or more potential chromatin fibers and identifying sister chromatids of a homolog chromosome.

In another aspect, this disclosure provides a system for analyzing chromatins, comprising: a non-transitory, computer-readable memory; one or more processors; and a computer-readable medium containing programming instructions that, when executed by the one or more processors, configure the system to:
(a) obtain a fluorescence imaging dataset comprising a three-dimensional image stack generated using a plurality of fluorescent probes hybridizing to discrete genomic loci on one or more chromatins, wherein the image stack comprises a plurality of fluorescence signals, each corresponding to a set of fluorescent probes of the plurality of fluorescent probes;
(b) associate a plurality of nodes respectively with the plurality of fluorescence signals, wherein one node is assigned to one fluorescence signal;
(c) assign a locus order to each of the nodes according to the genomic coordinate on a reference genome of each of the nodes such that one or more candidate nodes are associated with each locus order, and assign coordinates corresponding to spatial coordinates of a genomic locus detected in fluorescence imaging to define a spatial position of each of the nodes;
(d) connect a first candidate node of a first locus order with a second candidate node of a second locus order to form an edge, wherein the second locus order is greater than the first locus order by one or more loci orders;
(e) determine an edge weight based on a DNA polymer model to define a probability—often assigned as the negative logarithm transformed value—of the edge being an actual physical connection between two genomic loci represented by the first candidate node and the second candidate node on the reference genome;
(f) repeat steps (d) to (e) for remaining candidate nodes of the first locus order and remaining candidate nodes of the second locus order;
(g) traverse candidate nodes of remaining loci orders by repeating steps (d) to (f) to form a plurality of paths, each representing a spatial configuration of a potential chromatin fiber;
(h) determine a sum of edge weights of all the edges traversed in each of the paths, wherein the sum of edge weights defines a physical likelihood of the potential chromatin fiber; and
(i) identify one or more potential chromatin fibers having the sum of edge weights greater than a physical likelihood threshold.

In some embodiments, determining the edge weight comprises comparing observed pairwise spatial distance between the first candidate node and the second candidate node with estimated pairwise spatial distance between the two genomic loci represented by the first candidate node and the second candidate node on a reference chromatin fiber.

In some embodiments, the estimated pairwise spatial distance between the two genomic loci on the reference chromatin fiber is calculated using a freely joined Gaussian chain model. In some embodiments, the edge weight is determined by:

$${w}_{t ; i}^{t + c ; j} = \frac{1}{{( 2 {π ( {S}_{t ; i}^{t + c ; j} )}^{2} )}^{3 / 2}} {e}^{- ( \frac{{( {R}_{t ; i}^{t + c ; j} )}^{2}}{2 {( {S}_{t ; i}^{t + c ; j} )}^{2}} )}$$

where R_{t;i}^{t+c;j} is a distance in nanometers between the ith node with locus order t to the jth node with locus order t+c;
wherein S_{t;i}^{t+c;j} is expanded as:

$${( {S}_{t ; i}^{t + c ; j} )}^{2} = {σ}_{t ; i}^{2} + {σ}_{t + c ; j}^{2} + \frac{2}{3} {l}_{p} τ {L}_{t ; i}^{t + c ; j}$$

where positional uncertainties of both the start locus σ_{t;i}² and end locus σ_{t+c;j}² are appended to the second moment

$$〈 {R}^{2} 〉 = \frac{2}{3} {l}_{p} τ {L}_{t ; i}^{t + c ; j} ,$$

where lₚ is persistence length of DNA in nanometers, x is a scaling factor that converts genomic distance in base pairs to spatial distance in nanometers, and L_{t;i}^{t+c;j} is the genomic distance in base pairs that separate the start locus v_{t;i} and end locus v_{t+c;j}.

In some embodiments, determining the edge weight comprises transforming the probability of the edge with a negative logarithm function into positive edge weights.

In some embodiments, the physical likelihood of the potential chromatin fiber is defined by:

$$CDF = {\prod }_{h = 1}^{H} {w}_{{v}_{h}}^{{v}_{h + 1}} , p = 〈 {v}_{1} , {v}_{2} … {v}_{H} 〉$$

for every node v visited on path p from source to sink, wherein CDF represents conformational distribution function which defines the physical likelihood.

In some embodiments, the system is configured to rank physical likelihoods of the potential chromatin fibers and identify a potential chromatin fiber having the maximum physical likelihood.

In some embodiments, the system is configured to find the shortest path from a starting node of the first locus order to an ending node of an end locus order for the genomic loci on the reference genome.

In some embodiments, the system is configured to generate an adjacency matrix for finding the shortest path. In some embodiments, finding the shortest path is performed by dynamic programming. In some embodiments, the dynamic programming comprises performing a Dijkstra operation to find a least-cost path.

In some embodiments, at step (c) the system is configured to assign to each of the nodes positional uncertainty in each spatial axis discovered from three-dimensional gaussian fitting.

In some embodiments, the second locus order is not immediately adjacent to the first locus order, such that one or more intervening locus orders are skipped for edge connection. In some embodiments, the system is configured to apply a gap penalty for the one or more intervening locus orders skipped.

In some embodiments, the fluorescence imaging dataset is obtained from a fluorescence in situ hybridization (FISH) procedure selected from sequential fluorescent in situ hybridization (seqFISH+), single-molecule fluorescent in situ hybridization (smFISH), multiplexed error-robust fluorescence in situ hybridization (MERFISH), multiplexed DNA fluorescence in situ hybridization (M-DNA-FISH), and whole-genome DNA seqFISH+ imaging.

In some embodiments, the fluorescence imaging dataset is obtained from the fluorescence in situ hybridization (FISH) procedure on a eukaryotic cell. In some embodiments, the cell is in interphase. In some embodiments, the fluorescence imaging dataset is obtained from the fluorescence in situ hybridization (FISH) procedure on non-condensed chromosomes.

In some embodiments, the discrete genomic loci have an interval of about 1 kb to about 10 Mb. In some embodiments, the discrete genomic loci can have unidentical, nonuniform intervals (eg. 1.1 Mb→2.5 Mb→0.6 Mb→3.2 Mb→ . . . ) that span a chromosome. In some embodiments, the sequence of unidentical, nonuniform intervals can be appropriated as a spatial barcode, analogous to alternating black and white stripes of different widths in traditional barcodes.

In some embodiments, the system is configured to: (1) prior to step (i), accept all the potential chromatin fibers, (2) perform an iterative search wherein nodes of each shortest path discovered are subtracted and rendered unavailable for other path traversals before searching for the next shortest path, until no likely paths below the physical likelihood threshold remain to be discovered, and (3) count the number of all physically likely potential chromatin fibers.

In some embodiments, the system is configured to perform clustering (e.g., k-means clustering) on one or more potential chromatin fibers to determine a spatial distribution of the one or more potential chromatin fibers in one or more locations of chromosome territory.

In some embodiments, the system is configured to perform density-based clustering on one or more potential chromatin fibers and identify sister chromatids of a homolog chromosome.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows spatial genome alignment of multiplexed DNA-FISH imaging data against a reference soft-polymer structural model of DNA. Spatial coordinates in three dimensions (x, y, z) of a signal detected from each imaged loci are abstracted as nodes in a graph, ordered by their appearance on the reference genome. The identities of loci are depicted as circles, squares, stars, crosses, and triangles, corresponding to signals belonging to the first, second, third, fourth, and fifth locus, respectively. Utilizing a freely jointed Gaussian chain model, the aligner estimates an expected spatial distance based on the genomic distance separating two loci. The observed spatial distance is compared to the observed distance, and an edge between two loci is connected weighted proportionally to physical likelihood. The aligner was used to find the shortest path through the adjacency matrix, which returns the sequence of spatial positions whose path length equates to the most likely polymer.

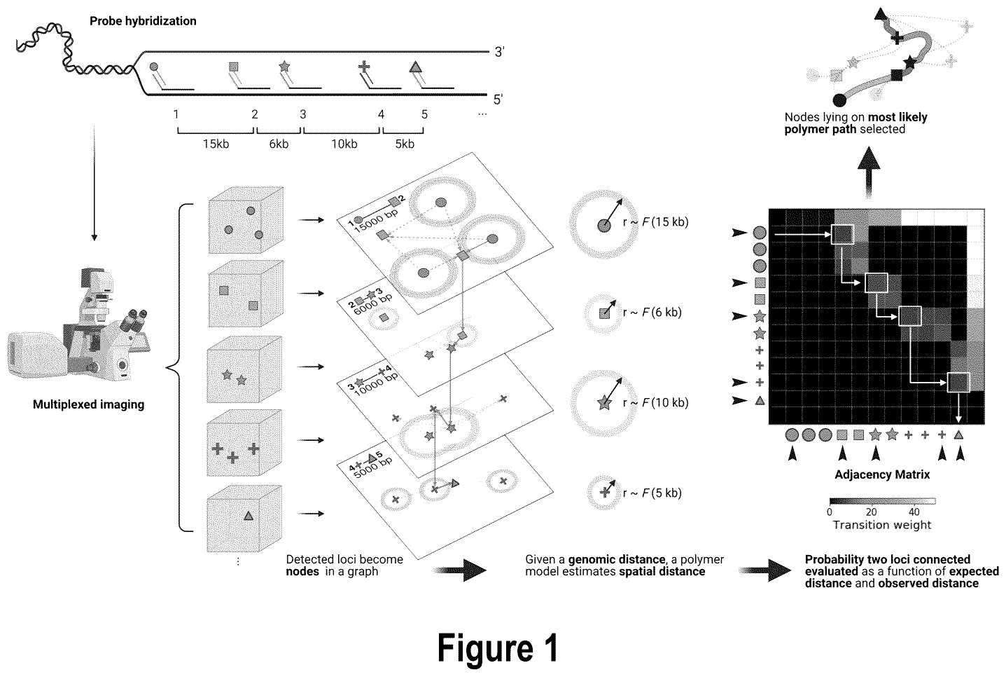

FIGS. 2A, 2B, 2C, and 2D show polymer fiber karyotyping of seqFISH+ chromatin imaging of mESC chr 1 at 1 Mb resolution. FIG. 2A shows an XY scatter plot of all detected fluorescence signals belonging to chr 1 loci. Loci identity are numbered adjacent to each signal, but the chromatin fiber identity is unknown. Multiple discrete signals attributed to the same locus (i.e., Left—Locus 16, Locus 17, Locus 18; Right—Locus 17) in close spatial proximity present ambiguity as to which signals are physically linked on the same chromatin fiber. FIG. 2B shows a polymer fiber karyotyping routine, iteratively applying spatial genome alignment to find all orthogonal sets of coordinates belonging to physically likely polymer fibers. Circles, triangles, crosses, and stars correspond to chromatin fiber ends of the first, second, third, and fourth chromatin fibers discovered, respectively. FIG. 2C shows the physical likelihood, also known as the conformational distribution function, of each polymer fiber discovered through the polymer fiber karyotyping routine. A circle, triangle, cross, and a star demarcate the conformational distribution function of the first, second, third, and fourth chromatin fibers discovered, respectively. FIG. 2D shows the output of polymer fiber karyotyping, delineating which signals are physically linked and lying on separate chromatin fibers. Circles, triangles, crosses, and stars correspond to chromatin fiber coordinates of the first, second, third, and fourth chromatin fibers discovered, respectively. Loci identities are numbered adjacent to each signal, and the chromatin fiber identity has been computed.

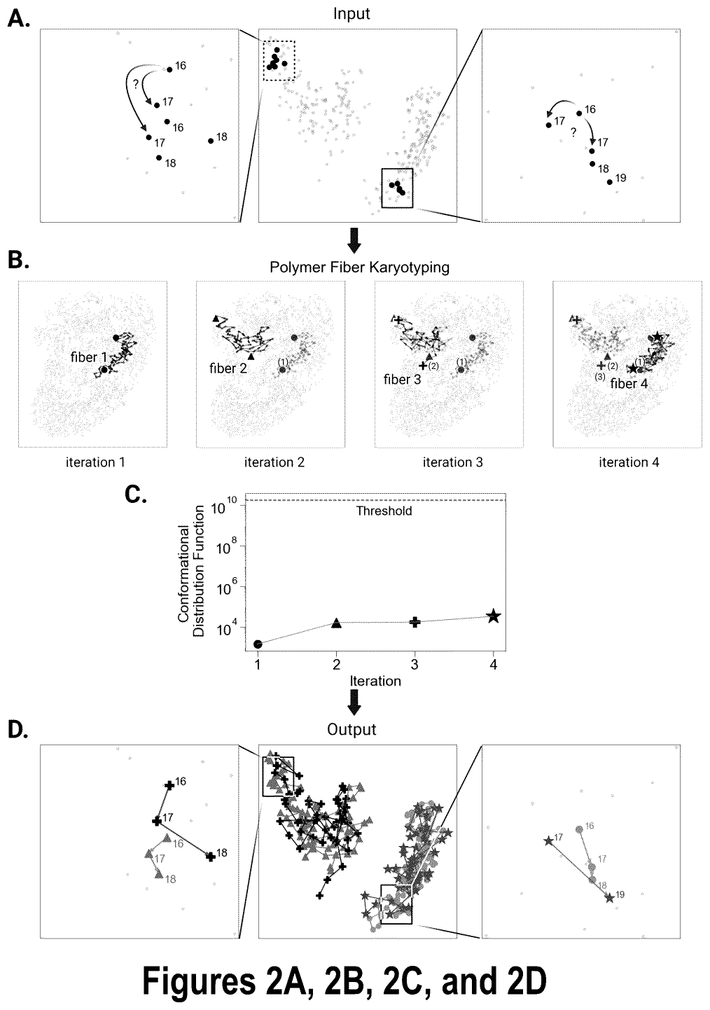

FIGS. 3A, 3B, and 3C show the benchmarking and performance of spatial genome alignment and polymer fiber karyotyping routines. FIG. 3A shows a heatmap of pairwise spatial distance between loci of all polymer fibers discovered from spatial genome alignment of seqFISH+ chromatin imaging mouse chr 1 at 1 Mb resolution (bottom left), juxtaposed to contact frequency from bulk proximity ligation assay or Hi-C binned at 1 Mb (top right). FIG. 3B shows a scatterplot of Spearman correlation between pairwise spatial distances (x-axis; log-normalized) imaged at 1 Mb resolution against Hi-C contact frequency (y-axis; log-normalized) binned at 1 Mb resolution. FIG. 3C shows a boxplot of assigned karyotype (x-axis) and total detected loci per chromosome, including spots omitted by spatial genome alignment of mESC chr 1 (y-axis). For every extra chromosome detected by polymer fiber karyotyping, a stepwise multiplicative increase of total detected loci (e.g., 1 chr—˜100 spots; 2 chr—˜200 spots; 3 chr—˜300 spots, etc.). Pearson correlation coefficient evaluates the strength of trend between detected loci and increase in assigned ploidy.

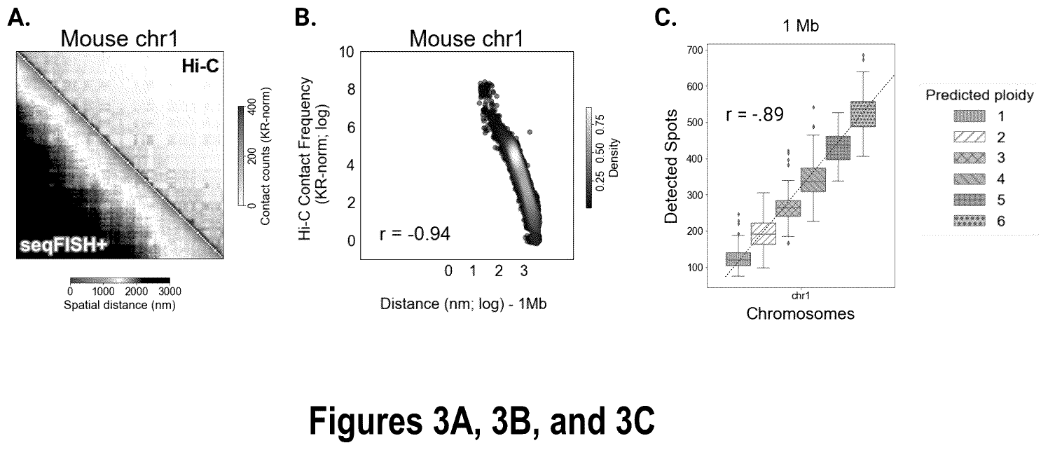

FIG. 4 shows an example application of the disclosed method. Probes of different genomic orders utilize the same fluorophore. Multiple loci are concurrently imaged in separate images, and the exact locus order is not immediately obvious from imaging. The disclosed method can be extended to inspect the observed spatial distance between pairs of spatial coordinates, and decode the sequential order of the locus order by finding the path that allows each observed pairwise spatial distance to match the expected spatial distance calculated from the reference genome.

### DETAILED DESCRIPTION OF THE INVENTION

This disclosure provides a novel method and system using a “spatial genome aligner” that parses true chromatin signals from noise by aligning signals to a DNA polymer model. This spatial genome aligner can efficiently reconstruct chromosome architectures from DNA-fluorescence in situ hybridization (DNA-FISH) data across multiple scales and determine chromosome ploidies de novo in interphase cells. Reprocessing of previous whole-genome chromosome tracing data with the disclosed method revealed the spatial aggregation of sister chromatids in S/G2 phase cells in asynchronous mouse embryonic stem cells and uncovered extranumerary chromosomes that remain tightly paired in post-mitotic neurons of the adult mouse cortex.

### Methods and Systems for Analyzing Chromatins

Accordingly, in one aspect, this disclosure provides a method for analyzing (e.g., tracing, karyotyping) chromatins, comprising:
(a) obtaining a fluorescence imaging dataset comprising a three-dimensional image stack generated using a plurality of fluorescent probes hybridizing to discrete genomic loci on one or more chromatins, wherein the image stack comprises a plurality of fluorescence signals, each corresponding to a set of fluorescent probes of the plurality of fluorescent probes;
(b) associating a plurality of nodes respectively with the plurality of fluorescence signals, wherein one node is assigned to one fluorescence signal;
(c) assigning a locus order to each of the nodes according to the genomic coordinate on a reference genome of each of the nodes such that one or more candidate nodes are associated with each locus order, and assigning coordinates corresponding to spatial coordinates of a genomic locus detected in fluorescence imaging to define a spatial position of each of the nodes;
(d) connecting a first candidate node of a first locus order with a second candidate node of a second locus order to form an edge, wherein the second locus order is greater than the first locus order by one or more locus orders;
(e) determining an edge weight based on a DNA polymer model to define a probability (e.g., often assigned as the negative logarithm transformed value) of the edge being an actual physical connection between two genomic loci represented by the first candidate node and the second candidate node on the reference genome;
(f) repeating steps (d) to (e) for remaining candidate nodes of the first locus order and remaining candidate nodes of the second locus order;
(g) traversing candidate nodes of remaining locus orders by repeating steps (d) to (f) to form a plurality of paths, each representing a spatial configuration of a potential chromatin fiber;
(h) determining a sum of edge weights of all the edges traversed in each of the paths, wherein the sum of edge weights defines a physical likelihood of the potential chromatin fiber; and
(i) identifying one or more potential chromatin fibers having the sum of edge weights greater than a physical likelihood threshold.

In some embodiments, the step of determining the sum of edge weights comprises calculating the physical likelihood of the potential chromatin fiber by determining the sum of negative logarithm transformed.

A “node” refers to a graph element that represents an entity (e.g., fluorescence signal) in a graph representation of a dataset (or data in general), such as a fluorescence imaging dataset. An “edge” refers to a graph element that represents a relationship between two nodes in a dataset in a graph representation of the dataset. As with nodes, edges may be categorized according to different types.

In some embodiments, one attribute of an edge may relate to a probability (e.g., weight) regarding the certainty of the relationship represented by the edge (e.g., a numerical value between 0 and 1, inclusive). When a dynamic programming algorithm (e.g., Dijkstra) is employed to find the shortest path, probabilities p are converted p to −log(p), so that the sum −log(p₁) −log(p₂) . . . −log(pₙ) is equal to the multiplicative product p₁*p₂ . . . * pₙ. A benefit of negative logarithm transformation is that with small numbers, the multiplicative product of many small numbers eventually becomes 0 (numerical underflow), and small numbers will no longer be distinguished and tracked. Negative logarithm transformation advantageously addresses this issue by converting small numbers into “large” numbers. Accordingly, the probability regarding the certainty of the relationship represented by the edge can be transformed with a negative logarithm into positive values between [1, ∞], with the benefits of: (1) preventing numerical underflow occurring in the multiplication of many small probabilities in the calculation of polymer likelihood; (2) transforming the calculation of polymer likelihood from the multiplication of probabilities to the equivalent sum of negative logarithm transformed probabilities; and (3) permitting the use of dynamic programming algorithms that compute the shortest path where the path length is the summation of edge weights traversed.

In some embodiments, the method comprises generating a directed acyclic graph (DAG) wherein chromatin fluorescent signals are abstracted as nodes in a DAG. The phrase “directed acyclic graph (DAG)” or “generalized directed acyclic graph (DAG),” as used herein, refers to a DAG structure (directed edges and no cycles) in which a child node can have multiple parents. A “tree” refers to a DAG structure in which each node can have only one parent node. A “graph” includes both trees and generalized DAGs.

Unless specifically stated otherwise, it is appreciated that throughout the disclosure, descriptions utilizing terms such as “obtaining,” “performing,” “receiving,” “computing,” “associating,” “assigning,” “traversing,” “calculating,” “determining,” “identifying,” “transforming,” “ranking,” “providing,” “transmitting,” or the like, refer to the action and processes of a computer system, or similar electronic computing device, that manipulates and transforms data represented as physical (or electronic) quantities within the computer system memories or registers or other such information storage, transmission or display devices.

In some embodiments, determining the edge weight comprises comparing observed pairwise spatial distance between two candidate nodes with estimated pairwise spatial distance between the two genomic loci represented by the candidate nodes on a reference chromatin fiber. In some embodiments, determining the edge weight comprises comparing observed pairwise spatial distance between the first candidate node and the second candidate node with estimated pairwise spatial distance between the two genomic loci represented by the first candidate node and the second candidate node on a reference chromatin fiber.

In some embodiments, the estimated pairwise spatial distance between the two genomic loci on the reference chromatin fiber is calculated using a freely joined Gaussian chain model, as described further below.

In some embodiments, the edge weight is determined by:

$${w}_{t ; i}^{t + c ; j} = \frac{1}{{( 2 {π ( {S}_{t ; i}^{t + c ; j} )}^{2} )}^{3 / 2}} {e}^{- ( \frac{{( {R}_{t ; i}^{t + c ; j} )}^{2}}{2 {( {S}_{t ; i}^{t + c ; j} )}^{2}} )}$$

where R_{t;i}^{t+c;j} is a distance in nanometers between the ith node with locus order t to the jth node with locus order t+c;
wherein S_{t;i}^{t+c;j} is expanded as:

$${( {S}_{t ; i}^{t + c ; j} )}^{2} = {σ}_{t ; i}^{2} + {σ}_{t + c ; j}^{2} + \frac{2}{3} {l}_{p} τ {L}_{t ; i}^{t + c ; j}$$

where positional uncertainties of both the start locus σ_{t;i}² and end locus σ_{t+c;j}² are appended to the second moment

$$〈 {R}^{2} 〉 = \frac{2}{3} {l}_{p} τ {L}_{t ; i}^{t + c ; j} ,$$

where lₚ is persistence length of DNA in nanometers, τ is a scaling factor that converts genomic distance in base pairs to spatial distance in nanometers, and L_{t;i}^{t+c;j} is the genomic distance in base pairs that separate the start locus v_{t;i} and end locus v_{t+c;j}.

In some embodiments, the physical likelihood of the potential chromatin fiber is defined by:

$$CDF = {\prod }_{h = 1}^{H} {w}_{{v}_{h}}^{{v}_{h + 1}} , p = 〈 {v}_{1} , {v}_{2} … {v}_{H} 〉$$

for every node v visited on path p from source to sink, wherein CDF represents conformational distribution function which defines the physical likelihood.

In some embodiments, determining the edge weight comprises transforming the probability of the edge with a negative logarithm function into positive edge weights. In some embodiments, the physical likelihood of the potential chromatin fiber is equivalently formulated as the sum of negative logarithm transformed edge weights. In some embodiments, the method comprises ranking physical likelihoods of the potential chromatin fibers and identifying a potential chromatin fiber having the maximum physical likelihood.

In some embodiments, the method comprises finding the shortest path from a starting node of the first locus order to an ending node of an end locus order for the genomic loci on the reference genome.

In some embodiments, the method comprises generating an adjacency matrix for finding the shortest path. For example, the method may use the adjacency matrix to represent edge weights of edges in a DAG graph.

In some embodiments, finding the shortest path is performed using a variety of dynamic programming techniques. Dijkstra's algorithm is an example of a dynamic programming approach that can be used to perform a search for the shortest path or the least-cost path between a starting node to an ending node according to the disclosure. Dynamic programming refers to methods of solving a complex problem by breaking it down into a collection of simpler subproblems, solving each of those subproblems only once, and storing their solutions (also referred to as “memoization”). As such, each memorized solution does not need to be re-solved the next time it is needed.

Dynamic programming algorithms can be used for optimization, such as finding the shortest paths between two nodes in a graph. For example, Dijkstra's algorithm can be used to solve the shortest path problem in a successive approximation scheme. To apply Dijkstra's algorithm, use a computer system to let the node deemed to be a starting node be called the initial node. Let the distance of node Y be the distance from the initial node to Y. Under Dijkstra's algorithm, the computer system will assign some initial distance values and will try to improve them step by step. First, assign to every node a tentative distance value: set it to zero for our initial node and to infinity for all other nodes. Second, set the initial node as current. Mark all other nodes unvisited. Create a set of all the unvisited nodes called the unvisited set. Third, for the current node, consider all of its unvisited neighbors and calculate their tentative distances. Compare the newly calculated tentative distance to the current assigned value and assign the smaller one. Otherwise, keep the current value. Fourth, when all of the neighbors of the current node have been considered, mark the current node as visited and remove it from the unvisited set. A visited node will never be checked again. Fifth, if the destination node has been marked visited (when planning a route between two specific nodes) or if the smallest tentative distance among the nodes in the unvisited set is infinity (when planning a complete traversal; occurs when there is no connection between the initial node and remaining unvisited nodes), then stop. The algorithm has finished. Sixth and finally, otherwise, select the unvisited node that is marked with the smallest tentative distance, set it as the new “current node,” and go back to the third step. More information regarding the use of Dijkstra's algorithm in a dynamic programming context can be found in Sniedovich, 2006, Dijkstra's algorithm revisited: the Dynamic Programming Connexion, Control and Cybernetics 25(3), the contents of which are incorporated by reference.

In some embodiments, the method comprises at step (c) assigning the coordinates corresponding to the spatial coordinates of the genomic locus comprises assigning to each of the nodes positional uncertainty in each spatial axis discovered from three-dimensional gaussian fitting.

In some embodiments, the second locus order is not immediately adjacent to the first locus order, such that one or more intervening locus orders are skipped for edge connection. In some embodiments, the method comprises applying a gap penalty for the one or more intervening locus orders skipped.

In some embodiments, the fluorescence imaging dataset is obtained from a fluorescence in situ hybridization (FISH) procedure selected from sequential fluorescent in situ hybridization (seqFISH+), single-molecule fluorescent in situ hybridization (smFISH), multiplexed error-robust fluorescence in situ hybridization (MERFISH), multiplexed DNA fluorescence in situ hybridization (M-DNA-FISH), and whole-genome DNA seqFISH+ imaging.

Nucleic acid hybridization techniques are based upon the ability of a single-stranded oligonucleotide probe to base-pair, i.e., hybridize, with a complementary nucleic acid strand. Exemplary in situ hybridization procedures are disclosed in U.S. Pat. No. 5,225,326, the entire contents of which are incorporated herein by reference. Fluorescence in situ hybridization refers to a nucleic acid hybridization technique that employs a fluorophore-labeled probe to specifically hybridize to and thereby facilitate visualization of a target nucleic acid. Such methods are well known to those of ordinary skill in the art and are disclosed, for example, in U.S. Pat. No. 5,225,326; U.S. patent application Ser. No. 07/668,751; PCT WO 94/02646, the entire contents of which are incorporated herein by reference. In general, in situ hybridization is useful for determining the distribution of a nucleic acid in a nucleic acid-containing sample, such as is contained in, for example, tissues at the single cell level. Such techniques have been used for karyotyping applications, as well as for detecting the presence, absence and/or arrangement of specific genes contained in a cell. However, for karyotyping, the cells in the sample typically are allowed to proliferate until metaphase (or interphase) to obtain a “metaphase-spread” prior to attaching the cells to a solid support for performance of the in situ hybridization reaction.

In some embodiments, the fluorescence imaging dataset is obtained from the fluorescence in situ hybridization (FISH) procedure on a eukaryotic cell. In some embodiments, the discrete genomic loci have an interval of about 1 kb to about 10 Mb (e.g., 5 kb, 10 kb, 15 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, 1 Mb, 2 Mb, 4 Mb, 6 Mb, 8 Mb, 10 Mb). In some embodiments, the discrete genomic loci can have unidentical, nonuniform intervals (e.g., 1.1 Mb→2.5 Mb→0.6 Mb→3.2 Mb→ . . . ) that span a chromosome. In some embodiments, the sequence of unidentical, nonuniform intervals can be appropriated as a spatial barcode, analogous to alternating black and white stripes of different widths in traditional barcodes. At time of imaging, multiple loci can be simultaneously imaged and abstracted as nodes with ambiguous locus order. The present method can be adapted to decode the locus order of simultaneously imaged spatial positions, inspecting the relative pairwise spatial distances and matching the observed distance with the most likely genomic distance in order to uncover its sequential order on the reference genome.

In another aspect, this disclosure additional provides a method for karyotyping a genome of a cell. The term “karyotype” refers to the genomic characteristics, e.g., the number and structure of the chromosomes, of an individual cell or cell line of a given species, e.g., as defined by both the number and morphology of the chromosomes. Typically, the karyotype is presented as a systematized array of prophase or metaphase (or otherwise condensed) chromosomes from a photomicrograph or computer-generated image. Alternatively, interphase chromosomes may be examined as histone-depleted DNA fibers released from interphase cell nuclei. In one embodiment, the karyotyping methods as disclosed are also used to determine copy number polymorphisms in a test cell or a test genome.

The existing methods using FISH imaging to karyotype are non-multiplexed. They label a chromosome only a few times or just once. If it has sufficient brightness, then it is considered a detection. There is no consideration of error. Accordingly, the existing methods lack detection confidence and are more sensitive to imaging artifacts such as off-target hybridization and failed hybridization. As a result, the existing methods are limited to cells in metaphase when chromosomes are condensed. They generally require lysing the cells to release DNA, which leads to cross-contamination of chromosomes from different cells.

In contrast, multiplexed FISH repeatedly labels the same chromosome multiple times at different locations. Such repeat labeling of the same spot many times leads to greater detection confidence. Unlike FISH imaging-based karyotyping, the disclosed method requires that spots not only have sufficient brightness, but also are spaced at just the right intervals (e.g., distances).

In addition, the existing methods using sequencing to karyotype cells are limited to observing relative fold changes of sequences mapped to each chromosome. They cannot distinguish the spatial organization of chromosomes, such as the inference of sister chromatids which are spatially observed to pair. This bears clinical significance as the sequencing detection of ie. an extranumerary third chromosome in a diploid cell may erroneously be classified an aneuploid cell, when it may also be an asynchronously replicating sister chromatid of an otherwise dividing euploid cell. Additionally, sequencing-based karyotyping methods suffer from poor detection efficiency as ie. a single diploid cell has only two copies of DNA, providing little starting material for sequencing. Current sequencing-based methods therefore lack single-cell sensitivity, subject to poor genome coverage and sequence amplification bias. Consequently, karyotyping results from the existing methods are often inaccurate and not reliable.

Compared to the existing methods, the method for karyotyping as disclosed herein are advantageous in several aspects, including: (a) the method can karyotype cells in all phases, such as those outside of metaphase (e.g., non-condensed, interphase chromosomes); (b) the method can karyotype chromosomes in intact cells without lysing cells to release chromosomes from the cells (e.g., directly inside an intact nucleus using imaging), either as cultured cells or cells embedded in intact tissue; (c) the method can karyotype chromosomes without depleting histones (e.g., directly inside an intact nucleus using imaging); (d) the method can karyotype chromosomes from multiplexed DNA-FISH imaging with high detection specificity by disambiguating true signal from noise using a polymer physics model; (e) the method can resolve patterns of spatial organization of chromosomes such as discerning sister chromatids without sister chromatid specific labeling; (f) the method can karyotype cells with single-cell accuracy without signal dropout and sequence bias.

Accordingly, this disclosure additionally provides a method for karyotyping a genome of a cell by directly karyotyping inside an intact nucleus irrespective of cell phase (e.g., interphase). The method eliminates the need of releasing DNA from the nucleus or compaction during metaphase. The method achieves single-cell karyotyping sensitivity, and the method can also identify spatial patterns of extranumerary chromosomes such as spatially proximal paired sister chromatids.

In some embodiments, the cell is in interphase. In some embodiments, the fluorescence imaging dataset is obtained from the fluorescence in situ hybridization (FISH) procedure on non-condensed chromosomes. In some embodiments, the method comprises: prior to step (i), (a) accepting all the potential chromatin fibers, (b) performing an iterative search wherein nodes of each shortest path discovered are subtracted and rendered unavailable for other path traversals before searching for the next shortest path, until no likely paths below the physical likelihood threshold remain to be discovered, and (c) counting or karyotyping the number of all physically likely potential chromatin fibers.

In another aspect, this disclosure additional provides a method for determining a spatial distribution of one or more potential chromatin fibers in one or more locations of chromosome territory. In some embodiments, the method comprises performing clustering (e.g., k-means clustering, hierarchical clustering, mean shift clustering, or a combination thereof) on one or more potential chromatin fibers to determine a spatial distribution of the one or more potential chromatin fibers in one or more locations of chromosome territory.

“K-Means clustering” refers to an unsupervised learning technique used to determine a mean of data (e.g., attribute vectors) in a cluster based on a distance (e.g., graph edit distance) and a centroid (median graph). In K-means clustering, data points (e.g., attribute vectors) may be partitioned into k clusters where each data point is associated with a cluster with the nearest mean. The mean serves as a prototype of the associated cluster. Agglomerative clustering starts by considering each data point (e.g., attribute vector) as a “cluster” and then merging clusters hierarchically.

“Hierarchical clustering” refers to the building (agglomerative) or break up (divisive), of a hierarchy of clusters. The traditional representation of this hierarchy is a dendrogram, with individual elements at one end and a single cluster containing every element at the other. Agglomerative algorithms begin at the leaves of the tree, whereas divisive algorithms begin at the root. Methods for performing hierarchical clustering are well known in the art. Hierarchical clustering methods have been widely used to cluster biological samples based on their gene expression patterns and derive subgroup structures in populations of samples in biomedical research (Bhattacharjee et al., 2001; Hedenfalk et al., 2003; Sotiriou et al., 2003; Wilhelm et al., 2002). “Agglomerative hierarchical clustering” refers to clustering techniques that produce a hierarchical clustering by starting with each point as a singleton cluster and then repeatedly merging the two closest clusters until a single, all-encompassing cluster remains. Agglomerative hierarchical clustering cannot be viewed as globally optimizing an objective function. Instead, agglomerative hierarchical clustering techniques use various criteria to decide locally, at each step, which clusters should be merged (or split for divisive approaches). This approach yields clustering algorithms that avoid the difficulty of attempting to solve a hard combinatorial optimization problem. Furthermore, such approaches do not have problems with local minima or difficulties in choosing initial points. Of course, the time complexity of O(m² log m) and the space complexity of O(m²) are prohibitive in many cases. Agglomerative hierarchical clustering algorithms tend to make good local decisions about combining two clusters since they can use information about the pair-wise similarity of all points. However, once a decision is made to merge two clusters, it cannot be undone at a later time. This approach prevents a local optimization criterion from becoming a global optimization criterion.

In another aspect, this disclosure additional provides a method for identifying sister chromatids of a homolog chromosome. In some embodiments, the method comprises performing density-based clustering on one or more potential chromatin fibers and identifying sister chromatids of a homolog chromosome.

“Density-based clustering” refers to techniques that map data based on an evaluation criterion, form clusters of the data included in regions of relatively high density, and identify data in regions of relatively low density as outliers (e.g., noise, etc.).

The present disclosure also provides a system and a computer program product. The computer program product may include a computer-readable storage medium (or media) having computer-readable program instructions thereon for causing a processor to carry out aspects of the present disclosure.

Accordingly, in another aspect, this disclosure also provides a system for analyzing chromatins, comprising: a non-transitory, computer-readable memory; one or more processors; and a computer-readable medium containing programming instructions that, when executed by the one or more processors, configure the system to:
(a) obtain a fluorescence imaging dataset comprising a three-dimensional image stack generated using a plurality of fluorescent probes hybridizing to discrete genomic loci on one or more chromatins, wherein the image stack comprises a plurality of fluorescence signals, each corresponding to a set of fluorescent probes of the plurality of fluorescent probes;
(b) associate a plurality of nodes respectively with the plurality of fluorescence signals, wherein one node is assigned to one fluorescence signal;
(c) assign a locus order to each of the nodes to define an order of a gene in a 5′ to 3′ direction on a genomic locus on a reference genome such that one or more candidate nodes are associated with each locus order, and assign coordinates corresponding to spatial coordinates of a genomic locus detected in fluorescence imaging to define a spatial position of each of the nodes;
(d) connect a first candidate node of a first locus order with a second candidate node of a second locus order to form an edge, wherein the second locus order is greater than the first locus order by one or more locus orders;
(e) determine an edge weight based on a DNA polymer model to define a probability (e.g., often assigned as the negative logarithm transformed value) of the edge being an actual physical connection between two genomic loci represented by the first candidate node and the second candidate node on the reference genome;
(f) repeat steps (d) to (e) for remaining candidate nodes of the first locus order and remaining candidate nodes of the second locus order;
(g) traverse candidate nodes of remaining locus orders by repeating steps (d) to (f) to form a plurality of paths, each representing a spatial configuration of a potential chromatin fiber;
(h) determine a sum of edge weights of all the edges traversed in each of the paths, wherein the sum of edge weights defines a physical likelihood of the potential chromatin fiber; and
(i) identify one or more potential chromatin fibers having the sum of edge weights greater than a physical likelihood threshold.

In some embodiments, the step of determining the sum of edge weights comprises calculating the physical likelihood of the potential chromatin fiber by determining the sum of negative logarithm transformed.

In some embodiments, determining the edge weight comprises comparing observed pairwise spatial distance between the first candidate node and the second candidate node with estimated pairwise spatial distance between the two genomic loci represented by the first candidate node and the second candidate node on a reference chromatin fiber.

In some embodiments, the estimated pairwise spatial distance between the two genomic loci on the reference chromatin fiber is calculated using a freely joined Gaussian chain model. In some embodiments, the edge weight is determined by:

$${w}_{t ; i}^{t + c ; j} = \frac{1}{{( 2 {π ( {S}_{t ; i}^{t + c ; j} )}^{2} )}^{3 / 2}} {e}^{- ( \frac{{( {R}_{t ; i}^{t + c ; j} )}^{2}}{2 {( {S}_{t ; i}^{t + c ; j} )}^{2}} )}$$

where R_{t;i}^{t+c;j} is a distance in nanometers between the ith node with locus order t to the jth node with locus order t+c;
wherein S_{t;i}^{t+c;j} is expanded as:

$${( {S}_{t ; i}^{t + c ; j} )}^{2} = {σ}_{t ; i}^{2} + {σ}_{t + c ; j}^{2} + \frac{2}{3} {l}_{p} τ {L}_{t ; i}^{t + c ; j}$$

where positional uncertainties of both the start locus σ_{t;i}² and end locus σ_{t+c;j}² are appended to the second moment

$$〈 {R}^{2} 〉 = \frac{2}{3} {l}_{p} τ {L}_{t ; i}^{t + c ; j} ,$$

where lₚ is persistence length of DNA in nanometers, τ is a scaling factor that converts genomic distance in base pairs to spatial distance in nanometers, and L_{t;i}^{t+c;j} is the genomic distance in base pairs that separate the start locus v_{t;i} and end locus v_{t+c;j}.

In some embodiments, determining the edge weight comprises transforming the probability of the edge with a negative logarithm function into positive edge weights.

In some embodiments, the physical likelihood of the potential chromatin fiber is defined by:

$$CDF = {\prod }_{h = 1}^{H} {w}_{{v}_{h}}^{{v}_{h + 1}} , p = 〈 {v}_{1} , {v}_{2} … {v}_{H} 〉$$

for every node v visited on path p from source to sink, wherein CDF represents conformational distribution function which defines the physical likelihood.

In some embodiments, the system is configured to rank physical likelihoods of the potential chromatin fibers and identify a potential chromatin fiber having the maximum physical likelihood.

In some embodiments, the system is configured to find the shortest path from a starting node of the first locus order to an ending node of an end locus order for the genomic loci on the reference genome.

In some embodiments, the system is configured to generate an adjacency matrix for finding the shortest path. In some embodiments, finding the shortest path is performed by dynamic programming. In some embodiments, the dynamic programming comprises performing a Dijkstra operation to find a least-cost path.

In some embodiments, at step (c) the system is configured to assign to each of the nodes positional uncertainty in each spatial axis discovered from three-dimensional gaussian fitting.

In some embodiments, the second locus order is not immediately adjacent to the first locus order such that one or more intervening locus orders are skipped for edge connection. In some embodiments, the system is configured to apply a gap penalty for the one or more intervening locus orders skipped.

In some embodiments, the fluorescence imaging dataset is obtained from a fluorescence in situ hybridization (FISH) procedure selected from sequential fluorescent in situ hybridization (seqFISH+), single-molecule fluorescent in situ hybridization (smFISH), multiplexed error-robust fluorescence in situ hybridization (MERFISH), multiplexed DNA fluorescence in situ hybridization (M-DNA-FISH), and whole-genome DNA seqFISH+ imaging.

In some embodiments, the fluorescence imaging dataset is obtained from the fluorescence in situ hybridization (FISH) procedure on a eukaryotic cell. In some embodiments, the discrete genomic loci have an interval of about 1 kb to about 10 Mb. In some embodiments, the discrete genomic loci can have unidentical, nonuniform intervals (eg. 1.1 Mb→2.5 Mb→0.6 Mb→3.2 Mb→ . . . ) that span a chromosome. In some embodiments, the sequence of unidentical, nonuniform intervals can be appropriated as a spatial barcode, analogous to alternating black and white stripes of different widths in traditional barcodes.

In some embodiments, the system is configured to: (1) prior to step (i), accept all the potential chromatin fibers, (2) perform an iterative search wherein nodes of each shortest path discovered are subtracted and rendered unavailable for other path traversals before searching for the next shortest path, until no likely paths below the physical likelihood threshold remain to be discovered, and (3) count the number of all physically likely potential chromatin fibers.

In some embodiments, the system is configured to perform clustering (e.g., k-means clustering) on the one or more potential chromatin fibers to determine a spatial distribution of the one or more potential chromatin fibers in one or more locations of chromosome territory.

In some embodiments, the system is configured to perform density-based clustering on the one or more potential chromatin fibers and identify sister chromatids of a homolog chromosome.

FIG. 8 is a functional diagram illustrating a programmed computer system in accordance with some embodiments. As will be apparent, other computer system architectures and configurations can be used to perform the described methods. Computer system 800, which includes various subsystems as described below, includes at least one microprocessor subsystem (also referred to as a processor or a central processing unit (CPU) 806). For example, processor 806 can be implemented by a single-chip processor or by multiple processors. In some embodiments, processor 806 is a general purpose digital processor that controls the operation of the computer system 800. In some embodiments, processor 806 also includes one or more coprocessors or special purpose processors (e.g., a graphics processor, a network processor, etc.). Using instructions retrieved from memory 807, processor 806 controls the reception and manipulation of input data received on an input device (e.g., image processing device 803, I/O device interface 802), and the output and display of data on output devices (e.g., display 801).

Processor 806 is coupled bi-directionally with memory 807, which can include, for example, one or more random access memories (RAM) and/or one or more read-only memories (ROM). As is well known in the art, memory 807 can be used as a general storage area, a temporary (e.g., scratchpad) memory, and/or a cache memory. Memory 807 can also be used to store input data and processed data, as well as to store programming instructions and data, in the form of data objects and text objects, in addition to other data and instructions for processes operating on processor 806. Also, as is well known in the art, memory 807 typically includes basic operating instructions, program code, data, and objects used by the processor 806 to perform its functions (e.g., programmed instructions). For example, memory 807 can include any suitable computer-readable storage media described below, depending on whether, for example, data access needs to be bi-directional or uni-directional. For example, processor 806 can also directly and very rapidly retrieve and store frequently needed data in a cache memory included in memory 807.

A removable mass storage device 808 provides additional data storage capacity for the computer system 800, and is optionally coupled either bi-directionally (read/write) or uni-directionally (read-only) to processor 806. A fixed mass storage 809 can also, for example, provide additional data storage capacity. For example, storage devices 808 and/or 809 can include computer-readable media such as magnetic tape, flash memory, PC-CARDS, portable mass storage devices such as hard drives (e.g., magnetic, optical, or solid state drives), holographic storage devices, and other storage devices. Mass storages 808 and/or 809 generally store additional programming instructions, data, and the like that typically are not in active use by the processor 806. It will be appreciated that the information retained within mass storages 808 and 809 can be incorporated, if needed, in a standard fashion as part of memory 807 (e.g., RAM) as virtual memory.

In addition to providing processor 806 access to storage subsystems, bus 810 can be used to provide access to other subsystems and devices as well. As shown, these can include a display 801, a network interface 804, an input/output (I/O) device interface 802, an image processing device 803, as well as other subsystems and devices. For example, image processing device 803 can include a camera, a scanner, etc.; I/O device interface 802 can include a device interface for interacting with a touchscreen (e.g., a capacitive touch sensitive screen that supports gesture interpretation), a microphone, a sound card, a speaker, a keyboard, a pointing device (e.g., a mouse, a stylus, a human finger), a global positioning system (GPS) receiver, a differential global positioning system (DGPS) receiver, an accelerometer, and/or any other appropriate device interface for interacting with system 800. Multiple I/O device interfaces can be used in conjunction with computer system 800. The I/O device interface can include general and customized interfaces that allow the processor 806 to send and, more typically, receive data from other devices such as keyboards, pointing devices, microphones, touchscreens, transducer card readers, tape readers, voice or handwriting recognizers, biometrics readers, cameras, portable mass storage devices, and other computers.

The network interface 804 allows processor 806 to be coupled to another computer, computer network, or telecommunications network using a network connection as shown. For example, through the network interface 804, the processor 806 can receive information (e.g., data objects or program instructions) from another network, or output information to another network in the course of performing method/process steps. Information, often represented as a sequence of instructions to be executed on a processor, can be received from and outputted to another network. An interface card or similar device and appropriate software implemented by (e.g., executed/performed on) processor 806 can be used to connect the computer system 800 to an external network and transfer data according to standard protocols. For example, various process embodiments disclosed herein can be executed on processor 806 or can be performed across a network such as the Internet, intranet networks, or local area networks, in conjunction with a remote processor that shares a portion of the processing. Additional mass storage devices (not shown) can also be connected to processor 806 through network interface 804.

In addition, various embodiments disclosed herein further relate to computer storage products with a computer-readable medium that includes program code for performing various computer-implemented operations. The computer-readable medium includes any data storage device that can store data that can thereafter be read by a computer system. Examples of computer-readable media include, but are not limited to: magnetic media such as disks and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code as produced, for example, by a compiler, or files containing higher level code (e.g., script) that can be executed using an interpreter.

The computer system as shown in FIG. 8 is an example of a computer system suitable for use with the various embodiments disclosed herein. Other computer systems suitable for such use can include additional or fewer subsystems. In some computer systems, subsystems can share components (e.g., for touchscreen-based devices such as smartphones, tablets, etc., I/O device interface 802 and display 801 share the touch-sensitive screen component, which both detects user inputs and displays outputs to the user). In addition, bus 810 is illustrative of any interconnection scheme serving to link the subsystems. Other computer architectures having different configurations of subsystems can also be utilized.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “genome” refers to any set of chromosomes with the genes they contain. For example, a genome may include, but is not limited to, eukaryotic genomes and prokaryotic genomes. The term “genomic region” or “region” refers to any defined length of a genome and/or chromosome. Alternatively, a genomic region may refer to a complete chromosome or a partial chromosome. Further, a genomic region may refer to a specific nucleic acid sequence on a chromosome (i.e., for example, an open reading frame and/or a regulatory gene).

The term “chromosome” refers to a single chromosome copy, e.g., a single molecule of DNA of which there are 46 in a normal somatic cell; an example is ‘the maternally derived chromosome 2’. Chromosome may also refer to a chromosome type, e.g., 23 chromosomes in a normal human somatic cell; an example is ‘chromosome 18’. Chromosome may refer to either a full chromosome, or a segment or section of a chromosome.

Copies refers to the number of copies of a chromosome segment. It may refer to identical copies, or to non-identical, homologous copies of a chromosome segment wherein the different copies of the chromosome segment contain a substantially similar set of loci, and where one or more of the alleles are different. Note that in some cases of aneuploidy, such as the M2 copy error, it is possible to have some copies of the given chromosome segment that are identical as well as some copies of the same chromosome segment that are not identical.

The term “haplotype” refers to a combination of alleles at multiple loci that are typically inherited together on the same chromosome. Haplotype may refer to as few as two loci or to an entire chromosome, depending on the number of recombination events that have occurred between a given set of loci. A haplotype can also refer to a set of SNPs on a single chromatid that are statistically associated.

The term “chromatin” refers to a complex of molecules comprising DNA, RNA, and proteins. More specifically, chromatin refers to a protein-DNA complex that packages DNA in the nucleus of cells. The basic unit of chromatin is the nucleosome, which is composed of 146 base pairs of DNA wrapped around an octamer of histone proteins, and other biomolecules may be associated with this complex.

The term “eukaryotic cell” refers to a cell having a nucleus and other organelles enclosed in a membrane. Non-limiting examples of eukaryotic cells are cells found in plants, fish, zebrafish, mice, humans, yeast, dogs, cows, etc.

The term “probe” refers to a molecule that can be recognized by a particular target. In some embodiments, a set of probe may concentrate around a particular target and emanate together as one fluorescence signal. The term “hybridization” refers to the process in which two single-stranded polynucleotides bind non-covalently to form a stable double-stranded polynucleotide. The term “hybridization” may also refer to triple-stranded hybridization, which is theoretically possible. The resulting (usually) double-stranded polynucleotide is a “hybrid.” The proportion of the population of polynucleotides that forms stable hybrids is referred to herein as the “degree of hybridization.” Hybridization probes usually are nucleic acids (such as oligonucleotides) capable of binding in a base-specific manner to a complementary strand of nucleic acid. Such probes include peptide nucleic acids, as described in Nielsen et al., Science 254:1497-1500 (1991) or Nielsen Curr. Opin. Biotechnol., 10:71-75 (1999) (both of which are hereby incorporated herein by reference), and other nucleic acid analogs and nucleic acid mimetics. The hybridized probe and target may sometimes be referred to as a probe-target pair. Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. No. 5,800,992 to Fodor, et al.; U.S. Pat. No. 6,040,138 to Lockhart, et al.; and International App. No. PCT/US98/15151, published as WO99/05323, to Balaban, et al.), genotyping (U.S. Pat. No. 5,856,092 to Dale, et al.), or other detection of nucleic acids. The '992, '138, and '092 patents, and publication WO99/05323, are incorporated by reference herein in their entireties.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

Aspects of the present disclosure are described herein with reference to flowchart illustrations and/or block diagrams of methods, apparatus (systems), and computer program products according to embodiments of the invention. In some embodiments, the flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be understood that, although the terms “first,” “second,” etc., may be used herein to describe various elements, components, regions, layers and/or sections. These elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

As used herein, “plurality” means two or more. As used herein, a “set” of items may include one or more of such items.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The term “if may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in the subsequent EXAMPLES below.

### Spatial Genome Alignment Algorithm

Conceptually, each detected fluorescence signal was abstracted from a 3D image stack as a 4-D node v=(x, y, z, t). Here, (x, y, z) correspond to sub-pixel spatial coordinates of a genomic locus detected in imaging, with a resultant positional uncertainty (σₓ, σ_{y}, σ_{z}) in each spatial axis discovered from 3D Gaussian fitting. The fourth dimension, t, corresponds to an order of the gene on the reference genome, ordered by its genomic coordinate for every chromosome. For notation, v_{t;i} was used to refer to a node i with spatial position (xᵢ, yᵢ, zᵢ) corresponding to order t on the reference genome; there may be as many as nₜ detected nodes for a given locus order t.
1. Graph construction: a directed acyclic graph G=(V, E) was defined as follows:
   V={v_{t;i}}, 1≤i≤nᵢ, 1≤t≤T represents the set of nodes in the graph, for every candidate node i of a locus order t among nₜ candidates, for all T genes on the reference genome.

Due to signal dropout, there may be genes for which nₜ=0, in which case no nodes for the given order t are populated.
E={w_{t;i}^{t+c;j}=(v_{t;i}, v_{t+c;j})}, 1≤i≤nₜ, 1≤j≤n_{t+c}, 1≤t≤T, 1≤c≤C represents the set of all edges connecting ordered pairs of nodes, between every candidate node i of a locus order t among nₜ candidates, to every candidate node j of a locus order t+c among n_{t+c} candidates, for allowable skips 1≤c≤C, for all T genes on the reference genome.

Self-loops were disallowed by enforcing a lower bound on the skip parameter c≥1, such that no edges propagate from one node to a node of lesser locus order, or more explicitly, no edges from a node of order t connect to any node with order less than or equal to t+1. This prevents discovery of certain structural variants, such as inversions, translocations, and duplications, but helps restrict solutions to strictly the reference genome. Nodes were permitted to “look ahead” to downstream genes by skipping up to a permissible upper bound c≤C, scaled later by an affine gap penalty. This accounts for signal dropout resulting in false-negative signals, in which case all nodes of a given order t maybe false positives and must be skipped.
2. Calculate bond probabilities: The edges were weighed using a physical analogy of a polymer model of DNA. Namely, the freely jointed Gaussian chain model was utilized, wherein chemical bonds model connections between two monomers. Here, the discrete spatially resolved genomic locations are analogous to these monomers, connected on the same chromatin fiber. In this model, the spatial distance separating these two locations was modeled after a Gaussian distribution:

$${w}_{t ; i}^{t + c ; j} = \frac{1}{{( 2 {π ( {S}_{t ; i}^{t + c ; j} )}^{2} )}^{3 / 2}} {e}^{- ( \frac{{( {R}_{t ; i}^{t + c ; j} )}^{2}}{2 {( {S}_{t ; i}^{t + c ; j} )}^{2}} )}$$

where R_{t;i}^{t+c;j} is the distance in nanometers between the ith node with locus order t to the jth node with locus order t+c. S_{t;i}^{t+c;j} is expanded as:

$${( {S}_{t ; i}^{t + c ; j} )}^{2} = {σ}_{t ; i}^{2} + {σ}_{t + c ; j}^{2} + \frac{2}{3} {l}_{p} τ {L}_{t ; i}^{t + c ; j}$$

where the positional uncertainties of both the start locus σ_{t;i}² and end locus σ_{t+c;j}² were appended to the second moment

$$〈 {R}^{2} 〉 = \frac{2}{3} {l}_{p} τ {L}_{t ; i}^{t + c ; j} .$$

Here, lₚ is the persistence length of DNA in nanometers, τ is a scaling factor that converts genomic distance in base pairs to spatial distance in nanometers, and L_{t;i}^{t+c;j} is the genomic distance in base pairs that separate the start locus v_{t;i} and end locus v_{t+c;j}. The positional uncertainty was calculated separately for the start and end locus to accommodate chromatic aberrations, in which case loci imaged using different laser channels may have different localization errors. τL_{t;i}^{t+c;j} together reflects the contour length of the DNA polymer. Collectively, this allows a comparison of the observed spatial distance R_{t;i}^{t+c;j} that separate two loci with an estimated spatial distance parametrized as S_{t;i}^{t+c;j}. In this present study, τ was estimated by fitting a power-law function through pairwise spatial distance and genomic interval data to estimate a length scale of each base pair per nanometer.

In this manner, a traversal from chromosome start to end along this graph would accumulate a sequence of bond probabilities whose product reflects the physical likelihood of the discovered polymer:

$$CDF = {\prod }_{h = 1}^{H} {w}_{{v}_{h}}^{{v}_{h + 1}} , p = 〈 {v}_{1} , {v}_{2} ... {v}_{H} 〉$$

for every node v visited on path p from source to sink.

Each edge weight w_{t;i}^{t+1;j} was negative log normalized into positive edge weights whose additive sum is equivalent to the polymer CDF. Such transformation was carried out for several reasons: (a) this controls for numerical underflow in calculating the multiplicative product of small decimals; (b) this reframes the optimization objective from maximizing the likelihood function to minimizing the negative log likelihood; and (c) the CDF is now computed as a sum of edge weights, permitting the use of existing dynamic programming shortest-path algorithms solving for additive edge weights. From here, edge weights w was used to represent negative log normalized bond probabilities. To permit nodes to skip potential false positives and “look ahead” to downstream genes, penalty was applied to each bond skipped in the following manner:

$${w}_{t ; i}^{′ t + c ; j} = {γ}_{c - 1} {w}_{t ; i}^{t + c ; j}$$

where γ_{c-1} is a gap penalty scaled for every skip c. Adjacent nodes (c=1) were not penalized.
3. Initialize adjacency matrix: A single source and a single sink node were appended in the graph to allow gaps in the start and end of polymer alignment. From the graph G with total N=Σₜ₌₁^{T} nₜ nodes, an (N, N) adjacency matrix that was padded by an additional row with index 1 and column with index N+2 to an (N+2, N+2) matrix was constructed. The first row of the adjacency matrix was initialized with “pseudo”-bonds that enable up to the first K genes to be skipped. These edges linking an imaginary starting position to an observed position were weighted as:

$${w}_{1 ; i}^{k ; j} = \frac{1}{{( \frac{4 π}{3} {l}_{p} τ {L}_{t ; i}^{t + c ; j} )}^{3 / 2}} {e}^{- {( \frac{{α}^{2} τ L}{\frac{4}{3} {l}_{p}} )}^{2}} , 1 < k \leq K$$

where α scales an imaginary stretched genomic segment with an implicit skip penalty.

The last column of the adjacency matrix was also initialized with “pseudo”-bonds that enable up to the last K genes to be skipped. Similarly, the edges linking an observed position to an imaginary ending position is weighted as:

$${w}_{N - k ; i}^{N ; j} = \frac{1}{{( \frac{4 π}{3} {l}_{p} τ {L}_{t ; i}^{t + c ; j} )}^{3 / 2}} {e}^{- {( \frac{{α}^{2} τ L}{\frac{4}{3} {l}_{p}} )}^{2}} , 1 < k \leq K$$

4. Path finding: The shortest path was searched from source to sink in the graph using a dynamic programming algorithm. As all edge weights in this directed graph are non-negative, and the sum of traversed edges equate to the CDF of the traversed polymer, Dijkstra's shortest-path algorithm was utilized as a dynamic programming means for finding the most plausible polymer:

$$min {\sum }_{h = 1}^{H} {w}_{{v}_{h}}^{{v}_{h + 1}} , {p}^{*} = 〈 {v}_{1} , {v}_{2} ... {v}_{H} 〉$$

for every node v visited on the shortest path p*.

The worst-case time complexity is estimated to be:

$$Θ ( \vert E \vert + \vert V \vert log \vert V \vert ) = Θ ( \vert {\sum }_{t = 1}^{T} {n}_{t} {\sum }_{t + 1}^{t + C \leq T} {n}_{t} \vert + \vert {\sum }_{t = 1}^{T} {n}_{t} \vert log \vert {\sum }_{t = 1}^{T} {n}_{t} \vert )$$

### Polymer Fiber Karyotyping Algorithm

A routine that finds all possible polymers of a given chromosome on a cell-by-cell basis was developed. Chiefly, all polymers below a physical likelihood threshold were accepted. This threshold can be derived by scrambling the genomic intervals separating each probed locus, such that the observed genomic distance no longer abides by the expected distances. An iterative search was then performed, wherein nodes of each shortest path discovered were subtracted from graph G before searching for the next shortest path, until no likely paths below the physical likelihood threshold can be discovered (FIG. 7).

Spatially resolved fluorescent signal information for mouse genome-wide seqFISH+ probe sets were retrieved at multiple length scales (1 Mb, 25 kb), and for multiple cell types (mESC: https://zenodo.org/record/3735329; mouse cortical neurons: https://doi.org/10.5281/zenodo. 4708112). As previously stated, a power-law function was fitted through pairwise spatial distances of observed loci, plotted against its genomic-distance separation. From this power-law function, a parameter was estimated corresponding to nanometers per base pair, for every cell type, and for every mouse chromosome. To evaluate the performance of the parameter in its ability to recapitulate true chromatin structure, a hyperparameter search was performed. Using 10% of the total dataset, spatial genome alignment was performed and compared its median distance matrix to Hi-C or cell-type resolved Dip-C. The parameter that engendered the best fit for the final analysis was utilized.

With this spatial distance parameter, for every nucleus and for every chromosome, spatial genome alignment was iteratively performed until no physically plausible fibers could be discovered. Finally, the number of fibers discovered for every chromosome was counted to assign a chromosome copy number, producing a karyotype.

mESC Hi-C Data Analysis

To evaluate the spatial genome alignment results of mESCs, mESC Hi-C contact matrices from the 4DN Data Portal (experiment set 4DNESU4Y9CBF) were retrieved. Next, Straw (https://github.com/aidenlab/straw) was utilized to extract Knight-Ruiz normalized count matrices for every mouse. Whereas read counts can be evenly binned, the loci imaged by seqFISH+ spanned irregular intervals. To compare Hi-C with seqFISH+ imaging data, where the genomic distances separating each locus are irregular intervals, the following normalization was performed. For seqFISH+ imaging data, the imaged locus ordered 5′ to 3′ closest to each integer 1 Mb or 25 kb bin was kept, dropping all other loci to calculate a final distance matrix. For the corresponding Hi-C matrix, the reads at 1 Mb and 25 kb resolution, respectively, were binned, dropping the same bins removed from the seqFISH+ distance matrix. To assess spatial genome alignment accuracy, the imaging distance matrix was compared to its corresponding Hi-C matrix using the Spearman correlation coefficient.

### Excitatory Mouse Cortical Neuron Dip-C Data Analysis

To evaluate the spatial genome alignment results of excitatory mouse cortical neurons, cell-type resolved Dip-C contact matrices were retrieved from NCBI GEO (accession GSE162511). First, a dearth of multi-modal data that ideally allows concomitant cell type classification using one sequencing modality and proximity-ligation analysis on the same cell. In Dip-C, neuronal cell types were resolved by co-projecting NeuN+ neurons with bulk Hi-C sequencing of multiple cell types. Neurons co-clustering with Slc17a7+ cells delineating excitatory neurons were also classified as excitatory. It is possible some of these cells classified as excitatory may belong to broader cell types. In contrast, seqFISH+ imaging resolved both RNA and DNA in the nuclei, which allowed excitatory neuron markers (i.e., Slc17a7+, Neurod2) detected in RNA imaging to label cell types. Second, a difference in the mouse strains of the two datasets. Dip-C focused on an F1 cross with CAST/EiJ×C57BL/6J background, while the seqFISH+ imaging purely focused on C57BL/6J mice.

Amid these caveats, Straw was utilized to extract Knight-Ruiz normalized count matrices for every mouse chromosome. For the seqFISH+ imaging data, the imaged locus ordered 5′ to 3′ closest to each integer 1 Mb bin were kept, dropping all other loci to calculate a final distance matrix. For the corresponding Dip-C contact matrix, the reads at 1 Mb resolution were binned and dropped the same bins removed from the imaging distance matrix. To assess spatial genome alignment accuracy, the imaging distance matrix was compared to its corresponding Hi-C matrix using the Spearman correlation coefficient.

Using the same Dip-C dataset, haplotype-resolved reads—namely pre-processed ‘seg’ files—were inspected to evaluate read counts assigned to each haplotype. Any ambiguous or multi-contact read pairs were discarded, and cells were counted cells wherein one haplotype has nearly twice as many reads as the other, as a proxy for identifying copy number variations and the haplotype source of those copy number variations at the single neuron level.

### Benchmarking Against M-DNA-FISH Chromatin Imaging Protocol and Spot Selection Algorithms

In conjunction, M-DNA-FISH imaging of a 210-kb genomic region spanning the Sox2 locus (chr3:34,601,078-34,811,078) was analyzed based on prior work (Bintu, B., et al., 2018. Science, 362(6413)). All chromosome centers assigned to the 129 allele, which lacks the 7.5 kb tandem CTCF-binding sites (CBSs) inserted on the CAST allele, were considered. The previous expectation-maximization routine outlined in Su et al. (Su, J., et al., 2020. Cell, 182(6), pp. 1641-1659.e26.) and Huang et al. (Huang, H., et al., 2021. Nature Genetics, 53(7), pp.1064-1074) generates 10 candidate spots per locus, per putative chromosome in every nucleus, assuming a diploid cell line. Each candidate spot is assigned a score, derived as a combination of (a) fluorescence intensity, (b) proximity to chromosome center, and (c) agreement with moving average of previous loci positions. A candidate spot with a score of −1.5 or more is considered a high-quality spot for the E-M routine, with more negative scores tracking with decreasing quality. For spatial genome alignment, all candidate spots were fed with a much lower quality threshold score of −4, such that for every high-quality spot, there is also a low-quality spot. This extra noise was included and ignored the fluorescence intensity information to demonstrate the supreme utility and specificity of genomic distances as a spot selection criterion. The spatial genome alignment results and E-M tracing results were compared against Hi-C, which sequenced the control mouse chr 3 lacking the CBS insertion. The Spearman correlation between the discovered pairwise distances and pairwise Hi-C contact frequency for each of the two algorithms was also calculated.

### Homolog Assignment and Sister Chromatid Aggregation Analysis

In diploid cells, a density-dependent clustering algorithm DBSCAN (scikit-learn) was utilized to separate homologous chromosomes residing in separate chromosome territories.

In tetraploid cells, instead of utilizing density-based clustering to parse and assign homologs, a different approach was taken. Chromatin interaction patterns (i.e., separate homologs, compact homologs, separated sisters in tetraploid cells) were first assigned with DBSCAN to find spatially separable structures and classify tetraploid cells. To assign sister chromatids and, in turn, homologs, chromatin fibers were paired by the closest starting positions and assigned as sisters of the same homolog. This allowed sisters to be paired as part of the same homolog in tetraploid cells which had only one spatially dense cluster (i.e., compact homologs). In the setting of compact chromosomes where two homologs are spatially not separable, alternative pairing scenarios were accounted for, such as pairing by the telomeric ends. The spatial separation of each chromosome starts and ends were analyzed based on pairing by centromeric starts as well as pairing by telomeric ends and all possible alternative pairings.

### Example 2

Multiplexed fluorescence in situ hybridization (FISH) has emerged as a powerful approach for analyzing 3D genome organization, but it is eminently challenging to derive chromosomal conformations from noisy fluorescence signals. Tracing chromatin is not straightforward as chromosomes lack conserved shapes for reference checking whether an observed fluorescence signal belongs to a chromatin fiber or not. This disclosure provides a novel method and system using a “spatial genome aligner” that parses true chromatin signals from noise by aligning signals to a DNA polymer model. This spatial genome aligner can efficiently reconstruct chromosome architectures from DNA-fluorescence in situ hybridization (DNA-FISH) data across multiple scales and determine chromosome ploidies de novo in interphase cells. Reprocessing of previous whole-genome chromosome tracing data with the disclosed method revealed the spatial aggregation of sister chromatids in S/G2 phase cells in asynchronous mouse embryonic stem cells and uncovered extranumerary chromosomes that remain tightly paired in post-mitotic neurons of the adult mouse cortex.

It was reasoned that while the shape of chromatin fiber is highly variable, it is subject to spatial constraints dictated by polymer physics. In addition to considering optical quality of signals, the algorithm selects the true fluorescence spots corresponding to a DNA locus from a number of candidates by picking the one that best conforms to a reference polymer model of chromatin. Briefly, these restrictions are the genomic distances between two labeled loci, which should be proportional to their spatial separation. A polymer model was used to estimate an expected spatial distance given a genomic distance and compare the observed spatial distance in imaging to this estimated spatial distance as a test of physical likelihood (Yamakawa, H. & Yoshizaki, T. Helical Wormlike Chains in Polymer Solutions. (2016). doi:10.1007/978-3-662-48716-7). The accuracy of the spatial genome aligner was evaluated by comparing pairwise distances discovered by tracing against pairwise contact frequencies discovered by Hi-C. The spatial genome aligner can recapitulate patterns of chromatin organization found in Hi-C at multiple genomic length scales (5 kb, 25 kb, and 1 Mb). Moreover, the spatial genome aligner uncovered more chromatin fibers than previously reported in published datasets and discovered these extra fibers are in fact sister chromatids. It was shown that each pair of sister chromatids usually reside in a spatially separate chromosome territory, but in ˜2% of replicating cells, both pairs of sister chromatids coalesce to interact in one convergent territory. The spatial genome alignment was applied to previous chromatin tracing data generated from mouse cortical excitatory neurons, where patterns of spatial organization of extranumerary chromosomes inside the nucleus were uncovered.

### Spatial Genome Alignment

Chromosomes are linear, flexible polymers that take on convoluted structures inside the nucleus. One simple but robust model for the spatial configuration of flexible polymers is a Gaussian chain. In this model, the polymer is represented as a chain of successive monomers, linked by bonds of approximately constant length b. Each successive monomer is allowed to freely rotate with respect to each other. Transitioning from one monomer to another along the polymer chain is to take one step in a three-dimensional random walk. For any two monomers i and j on this chain, the probability they are separated by a distance R₁ follows a Gaussian distribution (hence, Gaussian chain):

$$P ( {R}_{ij} , n ) = \frac{1}{{( \frac{2 π}{3} {nb}^{2} )}^{3 / 2}} {e}^{- ( \frac{3 {R}_{ij}^{2}}{2 {nb}^{2}} )}$$

where n is the number of bonds, each of length b, separating two monomers i and j.

For a chain with N bonds, the likelihood of the entire chain (also known as the conformational distribution function; CDF) is the product of all bond probabilities on the chain:

$$Ψ ( \{ {R}_{N} \} ) = {\prod }_{n = 1}^{N} \frac{1}{{( \frac{2 π}{3} {b}^{2} )}^{3 / 2}} {e}^{- ( \frac{3 {R}_{n}^{2}}{2 {b}^{2}} )}$$

In multiplexed DNA-FISH experiments, entire chromosomes are labeled at discrete positions, analogous to discrete monomers on a Gaussian chain. Furthermore, these discrete loci are interspaced by regular genomic intervals (e.g., 1 Mb), akin to the constant bond length b that separates monomers on the model chain. It was hypothesized that at large genomic length scales, DNA conformation can be modeled with a Gaussian chain in which the bond length b can be estimated from the genomic distance separating two loci(Yamakawa, H. & Yoshizaki, T. Helical Wormlike Chains in Polymer Solutions. (2016). doi:10.1007/978-3-662-48716-7; Ross, B. & Wiggins, P. Physical Review E 86, (2012)):

nb²=2lₚτL
where lₚ is the persistence length of DNA, τ is genomic-to-spatial distance conversion factor (nanometers per base pair), and L is the genomic distance separating two loci. τL together represents the contour length along the DNA polymer separating two loci.

In a setting where multiple signals are detected for each of two genomic loci, it is ambiguous which pair of signals lies on the same chromatin fiber. This Gaussian chain model permits expressing the probability two discrete loci imaged are physically connected as a function of both the observed spatial distance and the expected spatial distance. Here, the expected spatial distance is derived from the known genomic distance separating two loci on a reference genome. In taking one step along the chromatin fiber, a fluorescence signal can be selected or omitted by identifying (if any) a pair of signals whose observed spatial separation is ideally congruent with its expected spatial separation. In tracing the entire chromatin fiber, the most likely polymer among imaged loci is one where the collective segment lengths along the chromatin fiber best aligns with its expected segment lengths. The optimization objective is therefore to find the sequence of spatially resolved genomic loci that maximizes the likelihood, or CDF, of the polymer traced.

Algorithmically, imaged chromatin fluorescent signals were first abstracted as nodes in a directed acyclic graph (DAG). The topological order of nodes is determined by the order of loci on the reference genome. Each node was connected to the adjacent nodes on the linear genome, with each directed edge emulating a polymer segment. For each directed edge, genomic distances separating the two imaged loci to estimate an expected spatial distance were utilized. Both the expected spatial distance and observed spatial distance between the two imaged loci were utilized to calculate a bond probability, assigned as the edge weight. Traversing the graph from beginning to end is to find a potential chromatin fiber. Keeping track and multiplying the edge weights traversed, the score of one path reflects its physical likelihood (CDF).

Operationally, the edge probabilities were transformed with a negative logarithm function into positive edge weights, such that the additive sum of edge weights reflects the polymer CDF. With this transformation, the optimization objective of maximizing likelihood is reframed as minimizing the sum of negative logarithm transformation of edge probabilities. In other words, the objective is to find the shortest path through the graph representation of the polymer. Using dynamic programming, the shortest path was found through the adjacency matrix of the polymer graph. To account for false positives and false negative imaged spots, all valid paths are explored with the option to “skip” a node permitted by a gap penalty. Since DNA loci from a chromosome must lie on the same chromatin fiber which cannot branch, finding the shortest path is to find the most probable polymer without physical discontinuity discoverable from data.

The spatial genome aligner was first benchmarked against the chromatin tracing strategy that connects adjacent genomic loci by converting tabulated distances into an ensemble contact frequency. Previously published seqFISH+ genome-wide chromatin tracing on mouse embryonic stem cells (mESC) was analyzed, tracing every mouse chromosome at ˜1 Mb resolution across 1160 single cells (Takei, Y., et al. Nature, 590(7845), pp. 344-350). Unlike in published work, detected loci were binned and tabulated to convert distances into an ensemble contact frequency, the spatial genome aligner resolves single-molecule chromatin fibers at single-cell resolution across multiple genome scales. Indeed, the spatial genome aligner traces points whose structures are commensurate with bulk Hi-C (1 Mb: Spearman corr=−0.9±0.04; 25 kb: Spearman corr=−0.85±0.04). The spatial genome aligner resolved large chromatin compartments imaged at 1 Mb intervals as well as finer, single-cell chromatin domains imaged at 25 kb intervals. At 25-kb resolution, local chromatin structure is often nonlinearly organized into topologically association domains (TADs), with sudden shifts in chromatin compaction. Because the polymer model is a freely rotating chain of flexible segments, it accommodates such abrupt changes in local topology not easily captured when tabulated in an ensemble fashion.

To evaluate the performance of spatial genome alignment on finer genomic length scales and on data from other chromatin imaging protocols, spatial genome alignment was performed on multiplexed DNA-FISH data of the Sox2 locus imaged at 5-kb resolution (Huang, H., et al., 2021. Nature Genetics, 53(7), pp.1064-1074.). A protocol based on sequential DNA-FISH (Bintu, B., et al., 2018. Science, 362(6413)) was adapted to label a 210-kb genomic region on mouse chr3, spanning both the Sox2 gene locus in the F123 hybrid mESC line and its super-enhancer 110 kb downstream. By sequentially imaging these loci and tracing the chromatin, promoter-enhancer contacts corralled within a TAD were visualized. When the spatial genome aligner was applied to this fine 5-kb resolution chromatin imaging experiment, the spatial genome aligner recapitulated the TAD found in this region, faithfully capturing known promoter-enhancer interactions.

The spatial genome aligner was additionally benchmarked with a published chromatin tracing algorithm. Previously, chromatin tracing on multiplexed DNA-FISH emphasized the optical quality of a fluorescence spot, a metric incorporating (a) brightness, (b) proximity to a chromosome center, and (c) relative agreement to a moving average of preceding and subsequent spots. An expectation-maximization (E-M) procedure then sequentially selected one spot with the highest quality for each chromatin locus, while iteratively updating its quality scores. In contrast, the spatial genome aligner introduces another metric into spot selection—physical constraints dictated by polymer physics—as a decision criterion for selecting spots. Compared to previously published E-M spot selection algorithms (Spearman corr=−0.73), the spatial genome aligner achieves similar accuracy with respect to Hi-C(Spearman corr=−0.76). Notably, the spatial genome aligner performs a global optimization that incorporates the relative positioning of all imaged loci rather than a local moving average, and does so in quasilinear time using dynamic programming. Taken together, the genomic distance separating imaged loci critically helped disambiguate spot selection. The spatial genome aligner was able to accurately resolve chromatin fibers at multiple lengths scales, on multiple datasets, and on different multiplexed FISH imaging modalities.

### Polymer Fiber Karyotyping

A nucleus may have multiple copies of a chromosome. Finding all copies has traditionally relied on identifying compact clusters of imaged loci, aggregating by chromatin fiber. A k-means approach of clustering assumes the ploidy of a cell is known beforehand (Wang, S., et al. Science 353, 598-602 (2016)), this approach is unable to accommodate copy number variations. For k=2 and ploidy n=1, k-means may inadvertently look for a non-existent second “phantom” chromosome. Conversely, for k=2 and ploidy n=3, k-means would fail to detect an entire chromosome altogether. A ploidy-agnostic approach of clustering, such as DBSCAN, relies on density of detected loci (Takei, Y., et al., 2021. Nature, 590(7845), pp. 344-350; Takei, Y., et al. Science 374, 586-594 (2021)). However, the density neighborhood parameter is difficult to tune. A large density neighborhood may inadvertently aggregate two spatially separable chromosomes as a single dense cluster, misassigning two separate homologs as one. A small density neighborhood may fracture an intact chromosome into separate partitions.

By contrast, the spatial genome aligner provides a density or ploidy independent framework for identifying chromatin fibers. All detected spatial coordinates of a chromosome and a reference genome to the spatial genome aligner were provided, tasking it to extend, if possible, the most likely path from chromosome start to end. Since the path length (CDF) of a putative polymer reflects the physical likelihood of a polymer, it was reasoned that the karyotypes of interphase cells can be obtained simply by counting all physically likely polymer fibers. First, a likelihood threshold was set by scrambling a simulated polymer model of a reference genome such that the observed spatial distances between genes no longer abides by the genomic intervals that separate them. Next, spatial genome alignment was iteratively applied, extending polymer paths from putative seeds and subtracting nodes visited by the shortest path before searching for the next shortest path, until no physically likely polymer path was discovered. In this manner, orthogonal sets of coordinates belonging to contiguous chromatin fibers with likelihood scores below the threshold were produced. This process is termed polymer fiber karyotyping (PFK).

Using chromatin tracing data spanning the mouse genome at ˜1 Mb intervals, spatial genome alignment was performed to discover all possible chromatin fibers in the mouse ES cells. Intuitively, a diploid cell should have half as many chromatin fibers as a tetraploid cell. It was reasoned this should also reflect in the total number of loci detected in a cell. Comparing the total detected fluorescence signals per chromosome in a cell to its assigned ploidy determined by PFK, a linear relationship was observed. Every incremental increase in ploidy is accompanied by a stepwise, multiplicative increase in the total number of detected loci. Building on this, the agreement of karyotype assigned by each chromosome was compared. Hierarchical clustering of karyotype assigned by each chromosome across 1160 cells shows three distinct clusters of cells whose karyotype are homogenously congruent for all 19 somatic chromosomes. Namely, cells proportionally fell into a 6:2:2 distribution of 2N:3N:4N: cells, respectively, matching a replicative profile of highly dividing mESC. Treating each chromosome as a separate agent for karyotyping, the karyotype agreement between different chromosomes was quantified using Cohen's kappa test. Pairwise comparisons of each chromosome against another show demonstrably significant agreement (kappa>=0.3), saving chromosome X. Although the spatial genome aligner had every opportunity to find as many fibers for chromosome X as it did for other somatic chromosomes, it found half as many copies in this male cell line. This confirms that the spatial genome aligner produces accurate cell karyotypes without supervision, and that it discriminates karyotype in interphase where even the human eye cannot distinguish true copy number.

Aggregation of Sister Chromatids and Homologous Chromosomes in Tetraploid mESC Cells

Of the putative 4N cells karyotyped by the spatial genome aligner, whether these are polyploid cells with four separable chromosomes before replication or diploid cells with two pairs of sister chromatids after replication was investigated. As sister chromatids are shown to be tightly paired in a parallel fashion, it was reasoned that if two chromatin fibers reside in the same spatial neighborhoods, they are likely sister chromatids of the same homolog. Density-based clustering was performed to assign fibers of every ploidy to homologs. Under a set density parameter, the majority of diploid cells had two spatially resolvable fibers singularly residing in different territories. Notably, under the same density parameter, the majority of tetraploid cells also had not four spatially resolvable fibers but rather two clusters of paired fibers that cannot be parsed by eyes or by known clustering algorithms.

To test if these paired fibers are indeed sister chromatids, the trans-fiber loci distances relative to the cis-fiber loci distances were examined. In agreement with published sister-chromatid sensitive Hi-C on Drosophila and human cell lines (Mitter, M., et al., 2020. Nature, 586(7827) pp. 1139-144; Oomen, M., et al. Nature Methods 17, 1002-1009 (2020)), paired fibers in mouse ES cells resolved by the spatial genome aligner are spatially coupled. Explicitly, a given locus of one sister chromatid is followed by the same locus on its attendant sister chromatid, faithfully “shadowing” each other (e.g., chr 1, locus 1: μ=2065.8 nm separation; 90% CI [1801.6, 2330.0]). The spatial distance between cis-fiber interactions is closer for smaller genomic distances but converges with trans-fiber interactions above 10 Mb. Given the parallel nature of pairing and recapitulation of sister-chromatid interactions found by sister chromatid sensitive Hi-C, it was concluded that the tetraploid cells were in fact replicated diploid cells exhibiting paired sister chromatids.

Canonically, homologous chromosomes are divorced from each other in the nucleus and are widely acknowledged to reside in separate territories (Cremer, T. & Cremer, C. Nature Reviews Genetics 2, 292-301 (2001)). Yet, of the 207 cells tetraploid for chr 1, two predominant patterns: three-quarters (146/207 cells) bearing two spatially separable clusters presumed to be different homologs (sep-hom), and notably, a quarter (49/207 cells) with all four fibers are coalesced (compact), were observed. A marginal population of cells (12/207 cells) with three or more separable structures corresponds to separated sister chromatids (sep-sis). In case the clustering density parameter had inadvertently grouped two separable homologs together, each putative compact 4N chr 1 was visually inspected. Notably, a significant proportion of the compact state (31/49 candidates), cumulatively 2.67% of the total cell population, has four chromatin fibers spatially intermingling and which cannot be separated by eyes.

To investigate why the newly replicated homolog chromosomes coalesce, parsing which two fibers belong to one homolog within the compact structure was performed. Spatial proximity prohibits clustering from separating homologs and assigning pairs of fibers as sisters. Since true sister pairings should involve two fibers shadowing each other, it was reasoned sisters can be assigned by proximity of a given locus on two fibers. There are two natural assignments: grouping the closest pairs by the starting locus (SA; mouse centromere), and grouping the closest pairs by the end locus (EA; mouse telomere). Spatial proximity may cause the spatial genome aligner to inadvertently select spots belonging to other fibers. Therefore, the centromere-centromere distances as well as telomere-telomere distances of the two remaining permutations were explored. Specifically, these permutations correspond to the best possible alternate pairing (alt1) as well as the remaining pairing (alt2), ranked in this order.

When replicated homologs reside in separate chromosome territories, it was found that the telomeres of putative sister chromatids grouped by their centromere (SA) are likely coupled. The mean distance separating telomeres of SA sisters (μ=3857.5 nm; 90% CI [3451.5, 4263.5]), compared to the distance between its centromere-telomere not known to interact (μ=4474.9 nm; 90% CI [4295.8, 4654.1]). In contrast, the next best alternative pairing has a telomere separation (μ=5767.4 nm; 90% CI [5342.2, 6192.6]), larger than two non-interacting loci. In the same manner, the centromeres of putative sisters grouped by their telomere (EA) are also tightly coupled. All other alternate pairings exhibit a spatial separation above that of two non-interacting loci.

When replicated homologs coalesce, putative sisters grouped by their centromere may lose pairing at their telomeres. The mean distance separating telomeres of SA sisters is similar (μ=3286.5 nm; 90% CI [2835.9, 3737.1]), compared to the distance between two non-interacting loci (μ=4310.9 nm; 90% CI [4110.2, 4511.7]). Curiously, the next best alternative pairing has a telomere separation (μ=2544.2 nm; 90% CI [2131.2, 2597.1]) closer than the SA assigned telomere distance. Should this be due to misassignment, then all three pairing scenarios should share a uniformly unpaired distance distribution with mean distances above coupling. Yet, there almost always exists an alternate pairing between putative homologs that theoretically should not interact. The same analysis on EA sisters confers an ambiguous result, likely due to this loss of pairing.

The tendency for cis-homolog coupling decreases moving away from the centromere, resulting in a “flare-up” of putative trans-homolog interactions near the telomere of chromosomes. Since seqFISH+ labels discrete genomic loci, the contiguous polymer physically linking imaged loci was not visualized. Additionally, although seqFISH+ probes do not discriminate homologs, the spatial alignment analysis in bulk indicates a possible loss of sister pairing towards the telomeric end and increased trans-homologous interaction within this compact 4N state. The cells were ordered along the same pseudotime axis as determined using previously published cell cycle markers (H4K20me1, H4K16ac). It was found that this compact 4N state is scattered throughout interphase leading up to S phase. Additionally, this compact 4N state is rarely synchronized across multiple chromosomes, and appears to occur stochastically. This indicates that trans-homologous interactions between replicated chromosomes are likely uncoordinated and not initiated by a particular cell-cycle checkpoint.

### Chromosomal Copy Number Variations in Mouse Cortical Neurons

Aneuploidy has previously been reported in the brain, by both FISH and single-cell sequencing (McConnell, M., et al. Science 342, 632-637 (2013)). This copy number variation is thought to underlie a functional diversity adequately supporting neural complexity (Cai, X., et al. Cell Reports 8, 1280-1289 (2014)). Spatial genome alignment was applied to whole-genome DNA seqFISH+ imaging of 701 fully segmented mouse neurons (Takei, Y., et al. Science 374, 586-594 (2021)), all lying in the center z-sections of female mouse cortexes derived from 3 biological replicates. Of these intact nuclei, excitatory neurons, the predominant cell type in this dataset (n=458/701 neurons), were further investigated.

With polymer fiber karyotyping, copy number variations in mouse excitatory neurons were observed. While 58.05±6.38% (mean, standard deviation) of a given chromosome is 2N, both deletions (1N: 13.82±3.03%) and duplications (3N: 19.68±5.08%) were identified. Two broad patterns in hierarchical clustering were detected—a group of largely diploid cells with multiple chromosome deletions, and another group of diploid cells with multiple chromosome duplications. The karyotyping results were validated by analyzing haplotype resolved single-cell Dip-C sequencing on mouse cortical neurons classified as excitatory. By counting total reads, as well as inspecting the relative fold change of reads assigned to the maternal vs. paternal haplotype, the majority of sequenced neurons have balanced haplotyped reads (|log 2| fold change≤0.8). Interestingly, 12.15±1.36% of every chromosome has twice as many reads of one haplotype as the other. Not only may this reflect copy number variations prevalent in cortical neurons, it also indicates the extranumerary chromosome is contributed by one haplotype.

Because single-cell sequencing affords relative copy numbers of reads and fails to capture the nuclear organization of the aneuploid cells, the spatial organization of these copy number variations was inspected. In studying chr X, where the inactive chromosome is distinguished from the active by RNA imaging of Xist, density-based clustering reveals that excitatory neurons with three chr X have predominantly two chromosome territories. One chromatin fiber is standalone, while the two remaining fibers are constituents of the same territory. Of the doubly occupied chromosome territories, two-thirds are devoid of any Xist signal, suggesting a preference for active chr Despite different gene dosage, no significant gene expression relative to diploid cells was detected, irrespective of chr X activation status. The majority of labeled genes show no significant change relative to elevated gene dosage.

Between fibers in the doubly-occupied chromosome territory, the active chr X fibers show some degree of pairing (Spearman r=0.26) at a locus-to-locus level with its attendant fiber. This manifests as a strong diagonal in the trans-fiber pairwise distance matrix, as well as a spatial distance distribution mimicking that of cis-fiber pairwise distances. The same cannot be said of the inactive chr X, whose double constituents bear little resemblance.

### DISCUSSION

This disclosure provides a spatial genome aligner for multiplexed DNA-FISH data. This framework resolves chromatin fibers from discretely labeled positions of genomic loci, amid noise and signal dropout. In the spatial genome aligner, each observed locus' spatial position is checked against a reference model of a polymer chain. This reference model, a Gaussian chain abstracting connections between imaged loci as bond probabilities, dictates that even a highly variable structure as DNA follows predictable patterns of distance separation between loci. The model accurately captures chromatin compartments and domains on multiple lengths scales and across different chromosomes.

The algorithm falls into an early lineage of spatial genome aligners that abstracts connections between loci as polymer segments and whose edge weights are proportional to physical likelihood. Chiefly, although a reference polymer structure can reconcile each individual locus' most likely spatial position using the forward-backward algorithm (Ross, B. & Wiggins, P. Physical Review E 86, (2012); Rabiner, L. and Juang, B., 1986. IEEE ASSP Magazine, 3(1), pp. 4-16), it was demonstrated the utility of dynamic programming to find the most plausible sequence of spatial positions discoverable. In other words, finding the shortest path in the graph representation is to find the most physically-likely polymer without any physical discontinuity. It was shown that finding the most likely contiguous polymer is instrumental in uncovering copy number variations at the single-cell level. Through iterative subtraction of shortest paths finds all valid polymers, sister chromatids otherwise mistakenly grouped as one chromosome fiber were recovered. A new form of karyotyping called polymer fiber karyotyping was thus proposed, which is density- or clustering-independent. Ascribing a physical likelihood to polymers enhances detection sensitivity of a copy number of a gene in concrete terms, paving the way for the study of copy number variations in interphase for which the expected copy number is unknown. For instance, the study of oncogene amplification in the setting of cancer heterogeneity is currently limited by reliance on compact alignment of probes in metaphase spreads (Wu, S., et al. Nature 575, 699-703 (2019)). It is also held back by uncertainty in measurement due to unknown true copy number post-oncogene amplification.

In addition, resolving discrete polymer fibers instead of tabulating chromosome positions uncovered interchromosomal interactions. Multiplexed FISH captures in high throughput native chromosome structures directly in an intact nucleus among a spectrum of different replicative states. Chromosomes undergo transformative structural change throughout the cell cycle, disassembling the interphase nucleus to condense into sister chromatids during mitosis—a process that has been intensively studied using proximity ligation sequencing (Rehen, S., et al. Journal of Neuroscience 25, 2176-2180 (2005)). And yet, to date, sister chromatid level interactions have been difficult to resolve from imaging. This is due, in part, to a lack of biochemical labeling that can discriminately label sisters which are identical in sequence. Here, a computational reconstruction was proposed relying on statistical mechanics to resolve sister chromatid interactions from fluorescence imaging. Sister chromatids were distinguished from a welter of imaged loci difficult to be discerned by the human eye. While the majority of replicated homologs are divorced and reside in separate territories, compact territories where all four sister chromatids of a given chromosome spatially aggregate were uncovered. In separate territories each sister is shadowed by its attendant sister, whereas in the compact territory sister chromatids can lose sister pairing and might even pair with the other homolog. This structure is evocative of a crossover event, which is thought to occur in mitotic cells at exceedingly rare frequencies (˜1-2%).

Multiplexed DNA-FISH affords an intimate look into the elusive inner realities of genomic mosaicism in the brain. The intranuclear spatial organization of copy number variations was chronicled, with single-cell sensitivity and at whole-genome scale, heretofore only reported as frequencies (McConnell, M., et al. Science 342, 632-637 (2013)). It was shown that in neurons with three copies of a chromosome, the extranumerary chromosome shares a chromosome territory with another chromatin fiber, preserving two chromosome territories in the nuclei. Within the doubly occupied territory, each chromatin fiber appears to shadow each other, evocative of sister chromatids previously imaged in dividing mESCs. It is possible that the extranumerary chromosome is the derivative of a non-disjunction event, occurring in a neuroprogenitor during development. Another possibility is that the extranumerary chromosome is a remnant of asynchronous replication, a vestige in a neuroprogenitor that failed to withdraw or complete its replication timing (Chess, A., et al. Cell 78, 823-834 (1994)).

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### Example 3

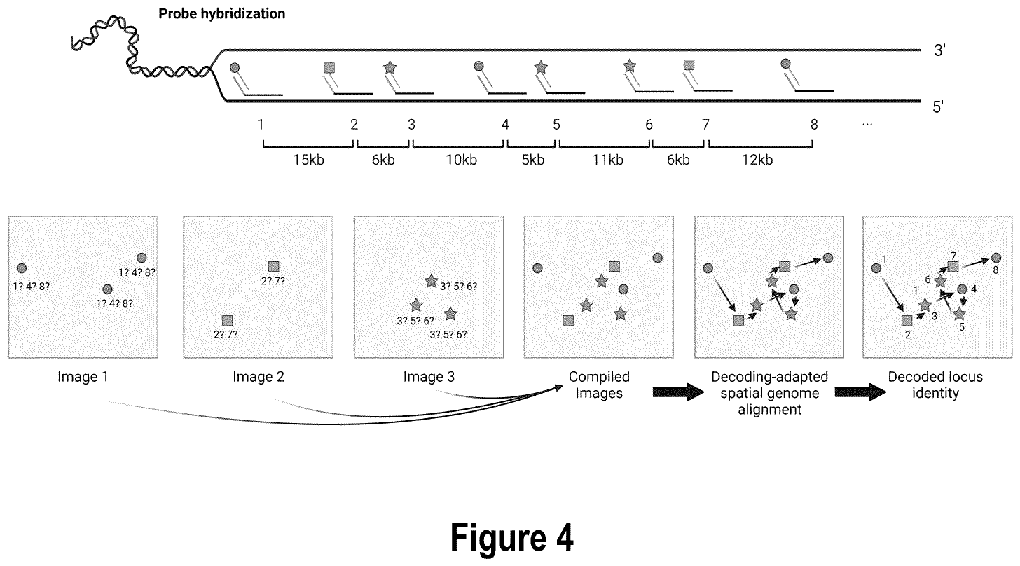

This disclosed method can be used for analyzing chromatins with fewer probes, which leads to reduced cost and makes it more scalable. As shown in FIG. 4, the same fluorophore can be utilized as probes of different genomic orders. Multiple loci are concurrently imaged in separate images, and the exact locus order is not immediately obvious from imaging. The disclosed method can be extended to inspect the observed spatial distance between pairs of spatial coordinates, and decode the sequential order of the locus order by finding the path that allows each observed pairwise spatial distance to match the expected spatial distance calculated from the reference genome.

1. A method of analyzing chromatins, comprising:
(a) obtaining a fluorescence imaging dataset comprising a three-dimensional image stack generated using a plurality of fluorescent probes hybridizing to discrete genomic loci on one or more chromatins, wherein the image stack comprises a plurality of fluorescence signals, each corresponding to a set of fluorescent probes of the plurality of fluorescent probes;
(b) associating a plurality of nodes respectively with the plurality of fluorescence signals, wherein one node is assigned to one fluorescence signal;
(c) assigning a locus order to each of the nodes according to the genomic coordinate on a reference genome of each of the nodes such that one or more candidate nodes are associated with each locus order, and assigning coordinates corresponding to spatial coordinates of a genomic locus detected in fluorescence imaging to define a spatial position of each of the nodes;
(d) connecting a first candidate node of a first locus order with a second candidate node of a second locus order to form an edge, wherein the second locus order is greater than the first locus order by one or more locus orders;
(e) determining an edge weight based on a DNA polymer model to define a probability of the edge being an actual physical connection between two genomic loci represented by the first candidate node and the second candidate node on the reference genome;
(f) repeating steps (d) to (e) for remaining candidate nodes of the first locus order and remaining candidate nodes of the second locus order;
(g) traversing candidate nodes of remaining locus orders by repeating steps (d) to (f) to form a plurality of paths, each representing a spatial configuration of a potential chromatin fiber;
(h) determining a sum of edge weights of all the edges traversed in each of the paths, wherein the sum of edge weights defines a physical likelihood of the potential chromatin fiber; and
(i) identifying one or more potential chromatin fibers having the sum of edge weights greater than a physical likelihood threshold.

2. The method of claim 1, wherein determining the edge weight comprises comparing observed pairwise spatial distance between the first candidate node and the second candidate node with estimated pairwise spatial distance between the two genomic loci represented by the first candidate node and the second candidate node on a reference chromatin fiber.

3. The method of claim 2, wherein the estimated pairwise spatial distance between the two genomic loci on the reference chromatin fiber is calculated using a freely joined Gaussian chain model.

4. The method of claim 1, wherein the edge weight is determined by:
$${w}_{t ; i}^{t + c ; j} = \frac{1}{{( 2 {π ( {S}_{t ; i}^{t + c ; j} )}^{2} )}^{3 / 2}} {e}^{- ( \frac{{( {R}_{t ; i}^{t + c ; j} )}^{2}}{2 {( {S}_{t ; i}^{t + c ; j} )}^{2}} )}$$
where R_{t;i}^{t+c;j} is a distance in nanometers between the ith node with locus order t to the jth node with locus order t+c;
wherein S_{t;i}^{t+c;j} is expanded as:
$${( {S}_{t ; i}^{t + c ; j} )}^{2} = {σ}_{t ; i}^{2} + {σ}_{t + c ; j}^{2} + \frac{2}{3} {l}_{p} τ {L}_{t ; i}^{t + c ; j}$$
where positional uncertainties of both the start locus σ_{t;i}² and end locus σ_{t+c;j}² are appended to the second moment
$$〈 {R}^{2} 〉 = \frac{2}{3} {l}_{p} τ {L}_{t ; i}^{t + c ; j} ,$$
where lₚ is persistence length of DNA in nanometers, τ is a scaling factor that converts genomic distance in base pairs to spatial distance in nanometers, and L_{t;i}^{t+c;j} is the genomic distance in base pairs that separate the start locus v_{t;i} and end locus v_{t+c;j}.

5. The method of claim 1, wherein determining the edge weight comprises transforming the probability of the edge with a negative logarithm function into positive edge weights.

6. The method of claim 1, wherein the physical likelihood of the potential chromatin fiber is defined by:
$$CDF = {\prod }_{h = 1}^{H} {w}_{{v}_{h}}^{{v}_{h + 1}} , p = 〈 {v}_{1} , {v}_{2} ... {v}_{H} 〉$$
for every node v visited on path p from source to sink, wherein CDF represents conformational distribution function which defines the physical likelihood.

7. The method of claim 1, comprising ranking physical likelihoods of the potential chromatin fibers and identifying a potential chromatin fiber having the maximum physical likelihood.

8. The method of claim 1, comprising finding the shortest path from a starting node of the first locus order to an ending node of an end locus order for the genomic loci on the reference genome.

9. The method of claim 8, comprising generating an adjacency matrix for finding the shortest path.

10. The method of claim 8, wherein finding the shortest path is performed by dynamic programming.

11. The method of claim 10, wherein the dynamic programming comprises performing a Dijkstra operation to find a least-cost path.

12. The method of claim 1, wherein at step (c) assigning the coordinates corresponding to the spatial coordinates of the genomic locus comprises assigning to the each of the nodes positional uncertainty in each spatial axis discovered from three-dimensional gaussian fitting.

13. The method of claim 1, wherein the second locus order is not immediately adjacent to the first locus order such that one or more intervening locus orders are skipped for edge connection.

14. The method of claim 13, comprising applying a gap penalty for the one or more intervening locus orders skipped.

15. The method of claim 1 procedure selected from sequential fluorescent in situ hybridization (seqFISH+), single-molecule fluorescent in situ hybridization (smFISH), multiplexed error-robust fluorescence in situ hybridization (MERFISH), multiplexed DNA fluorescence in situ hybridization (M-DNA-FISH), and whole-genome DNA seqFISH+ imaging.

16. The method of claim 15, wherein the fluorescence imaging dataset is obtained from the fluorescence in situ hybridization (FISH) procedure on a eukaryotic cell.

17. The method of claim 1, wherein the discrete genomic loci have a uniform interval of about 1 kb to about 10 Mb, or nonuniform and unidentical intervals between 1 kb to 10 Mb spanning the entire chromosome.

18. The method of claim 1, comprising:
prior to step (i), accepting all the potential chromatin fibers,
performing an iterative search wherein nodes of each shortest path discovered are subtracted and rendered unavailable for other path traversals before searching for the next shortest path, until no likely paths below the physical likelihood threshold remain to be discovered, and
counting the number of all physically likely potential chromatin fibers.

19. The method of claim 1, comprising performing k-means clustering on the one or more potential chromatin fibers to determine a spatial distribution of the one or more potential chromatin fibers in one or more locations of chromosome territory.

20. The method of claim 16, wherein the cell is in interphase, the cell lacks condensed chromosomes, the cell nucleus is intact without the release of chromosomes from cells, the cell nucleus is not depleted of histones, and/or the cell is imaged at single-cell resolution.

21. The method of claim 1, comprising performing density-based clustering on the one or more potential chromatin fibers and identifying sister chromatids of a homolog chromosome without differentially labeling the sister chromatid fiber in an experiment.

22. A system comprising one or more processors configured to implement the method of claim 1.

23-42. (canceled)
